(19) United States
(12) Patent Application Publication
(10) Pub. No.: US 2025/0144168 A1
(43) Pub. Date: May 8, 2025

(54) LANTHIPEPTIDES AND METHODS OF USE

(71) Applicant: OSTIA SCIENCES INC., Toronto (CA)

(72) Inventors: Abdelahhad Barbour, Toronto, ON (CA); Michael Glogauer, Toronto, ON (CA)

(21) Appl. No.: 18/837,439

(22) PCT Filed: Feb. 10, 2023
(86) PCT No.: PCT/CA2023/050180

§ 371 Date: Aug. 9, 2024

Related U.S. Application Data
(60) Provisional application No. 63/308,841, filed on Feb. 10, 2022.

(51) Int. Cl. A61K 38/16; A61P 31/04; C07K 14/315

(52) U.S. Cl. CPC A61K 38/164; A61P 31/04; C07K 14/315

(57) ABSTRACT

Compositions comprising a pre-SrnA1 peptide, an SrnA1 peptide, a pre-SrnA2 peptide, an SrnA2 peptide, a pre-SrnA4 peptide, a SrnA4, and other peptides and post-translationally modified versions thereof are taught. Also taught are methods of treating bacterial infections comprising administering a pharmaceutically acceptable amount of the composition to the patient with a bacterial infection caused by a pathogen from, for example, multi-drug resistant (MDR) Streptococcus pneumoniae, Streptococcus pyogenes, Streptococcus dysagalactiae, vancomycin-resistant Enterococcus faecium, Porphyromonas gingivalis, Tannerella forsythia, and MDR Enterococcus faecalis.

### FIELD OF THE INVENTION

The invention pertains to novel Salivaricin lanthipeptides, particularly, phosphorylated lanthipeptides, derived from the oral microbiome, and methods of using the same for protection against oral diseases and other indications.

### BACKGROUND OF THE INVENTION

Bacteria associated with the human host can either be commensals of low virulence or profoundly virulent microorganisms, which can cause extreme disease manifestation and, in many cases, death [1]. The current advancement in molecular microbiology revealed that commensals also produce metabolites that can regulate the host neutrophils and macrophages resulting in better innate immunity responses [2].

Although considerable research addressing significant microbial processes presently exist, including resistance to antimicrobials, virulence, and colonization, the mechanisms governing commensal-host communication in the oral microbiome and their symbiotic relationship to keep opportunistic pathogens in check remain poorly understood. The human oral microbiota is a dynamic and diverse ecological niche colonizing on distinct microenvironments, including the teeth' hard surfaces and the mucosa's epithelial surfaces [3]. Several processes bear the transition of a homeostatic equilibrium of the oral microbiome to a dysbiosis state. As the oral polymicrobial community evolves, microbial metabolites and host immune response can cause changes to the local environment that facilitate the outgrowth or over-representation of microorganisms associated with a dysbiotic state [4].

Differences in host immune responses, environmental factors, or nutrition can influence the polymicrobial composition and the meta-transcriptional landscape, which may increase the production of virulence determinants. Once a community has transitioned to a dysbiotic state, the structural stability of functionally specialized components will render the condition to persist for an extended period of time, and oral diseases are often chronic and slowly progressing [5, 6]. To prevent such a scenario, commensal oral microbes, which form the main constituents of the oral biofilm, possess diverse mechanisms to crowd out harmful microbes and communicate with the host cells to produce an immunological response that will result in the prevention of disease progression [7]. One of the earliest colonizers of the oral cavity is Streptococcus salivarius [8], which bestow the establishment of immune homeostasis and management of host inflammatory responses by inhibiting the activation of the NF-β pathway [9]. In addition to immunoregulation capacities, selected strains of this bacterium secrete a tandem array of ribosomally synthesized and post-translationally modified peptides (RiPPs) called salivaricins [10]. Though not conserved in many strains, most salivaricins belong to the lantibiotic class II of antibiotics. The term lanthipeptides is a short-hand denotation for lanthionine-containing peptides, and when lanthipeptides possess antimicrobial activities, we call them lantibiotics [11]. The increase in the bacterial whole genome sequencing projects resulted in increased numbers of characterized lanthipeptides and has led to the realization that their functions extend beyond their antimicrobial activities to now include antifungal [12], antiviral [13, 14] antinociceptive [15], antiallodynic functions [16] and host defense immunomodulation [17, 18].

Lantibiotics are RiPPs produced through a process that involves the dehydration of selected serine (Ser) and threonine (Thr) residues and the intramolecular addition of cysteine (Cys) thiols to the resulting unsaturated amino acids to produce lanthionine (Lan) and methyllanthionine (MeLan) rings, respectively [19, 20]. Dehydration during biosynthesis of lanthipeptides includes the phosphorylation of Thr and Ser residues by the lanthionine dehydratase enzyme followed by phosphate elimination with anti-stereoselectivity. Phosphorylated lanthipeptides are not observed during in vitro synthesis, suggesting that phosphate elimination is faster than dissociation of the intermediate phosphopeptide [20].

Novel lanthipeptides may provide advantageous therapeutic properties.

### SUMMARY OF THE INVENTION

According to one aspect of the invention is provided a composition comprising a pre-SrnA1 peptide having a sequence of SEQ ID NO.: 1, a SrnA1 peptide having a sequence of SEQ ID NO: 2, a pre-SrnA2 peptide having a sequence of SEQ ID NO.3, a SrnA2 peptide having a sequence of SEQ ID NO: 4, a pre-SrnA4 peptide having a sequence of SEQ ID NO.: 7, a SrnA4 peptide having a sequence of SEQ ID NO: 8, or post-translationally modified versions thereof.

In certain embodiments, the composition consists of SrnA1, SrnA2, and SrnA4, or post-translationally modified versions thereof.

In certain embodiments, the composition consists of post-translationally modified versions of SrnA1, SrnA2, and SrnA4.

In certain embodiments, the post translational modifications comprise phosphorylation.

In certain embodiments, the phosphorylation comprises a phosphorylation at Thr4 amino acid of each or all of SrnA1, SrnA2 and SrnA4.

In certain embodiments, the post translational modifications comprise a single phosphorylation at Thr4 amino acid of each or all of SrnA1, SrnA2 and SrnA4.

In certain embodiments, the post-translational modifications comprise one or more dehydrations.

In certain embodiments, the post-translational modifications comprise dehydrations at one or more of amino acids 9, 19 and 21 of each or all of SrnA1, SrnA2 and SrnA4.

In certain embodiments, the post-translational modifications comprise a 6-methyllanthionine ring between amino acids 9 and 14 (Abu9-S-Ala14), a 6-methyllanthionine ring between amino acids 19 and 24 (Abu19-S-Ala24), and a lanthionine ring between amino acids 21 and 31 (Ala21-S-Ala31) of each or all of SrnA1, SrnA2 and SrnA4.

In certain embodiments, the compositions are derived from peptides having sequences of SEQ ID NO:1, SEQ ID NO:3, and/or SEQ ID NO: 7.

In certain embodiments, the compositions are derived from an S. salivarius.

In certain embodiments, the compositions are derived from a SALI-10 strain of S. salivarius.

In certain embodiments, the SALI-10 is characterized by comprising a megaplasmid.

In certain embodiments, the megaplasmid has a size of 164 kb.

In certain embodiments, the megaplasmid has a sequence of SEQ ID NO.: 15.

In certain embodiments, the SALI-10 is characterized by comprising a gene sequence of SEQ ID NO.: 14.

In certain embodiments, the SALI-10 comprises an SrnM lanthionine synthetase having a sequence of SEQ ID NO:9.

According to a certain aspect of the present invention is provided a pharmaceutical composition comprising the composition and a pharmaceutically acceptable excipient or carrier.

According to a certain aspect of the present invention is provided a method of treating a bacterial infection comprising administering a pharmaceutically acceptable amount of the pharmaceutical composition to a patient.

In certain embodiments, the bacterial infection is caused by a pathogen selected from the group consisting of multi-drug resistant (MDR) Streptococcus pneumoniae, Streptococcus pyogenes, Streptococcus dysagalactiae, vancomycin-resistant Enterococcus faecium, Porphyromonas gingivalis, Tannerella forsythia, and MDR Enterococcus faecalis.

According to a certain aspect of the present invention is provided a method of treating Actinomyces graevenitzii and/or Granulicatella adiacens-associated pulmonary abscess in a patient in need thereof, comprising administering a pharmaceutically acceptable amount of the pharmaceutical composition to the patient.

According to a certain aspect of the present invention is provided a method of treating Bifidobacterium dentium associated caries in a patient in need thereof, comprising administering a pharmaceutically acceptable amount of the pharmaceutical composition to the patient.

According to a certain aspect of the present invention is provided a method of treating of S. pyogenes associated strep throat in a patient in need thereof, comprising administering a pharmaceutically acceptable amount of the pharmaceutical composition to the patient.

In certain embodiments, the administration is oral and the amount of commensal bacteria in the oral cavity is maintained or augmented.

According to a certain aspect of the present invention is provided a method of ameliorating oral health in a patient desirous thereof comprising administration of a pharmaceutically acceptable amount of the pharmaceutical composition to the oral cavity of the patient.

According to a certain aspect of the present invention is provided a method of modifying oral biofilm in an oral cavity of a patient desirous thereof by reducing disease-associated bacteria while maintaining number of essential commensals in said oral cavity, comprising administering a pharmaceutically acceptable amount of the pharmaceutical composition to the oral cavity of the patient.

According to a certain aspect of the present invention is provided a method of killing biofilm-associated bacteria in an oral cavity of a patient desirous thereof, comprising administering a pharmaceutically acceptable amount of the pharmaceutical composition to the oral cavity of the patient.

According to a certain aspect of the present invention is provided a method of modulating an anti-inflammatory response in a patient in need thereof, comprising administering a pharmaceutically acceptable amount of the pharmaceutical composition to the patient.

According to a certain aspect of the present invention is provided a method of reducing oral inflammation in a patient in need thereof, comprising administering a pharmaceutically acceptable amount of the pharmaceutical composition to the patient.

According to a certain aspect of the present invention is provided a method of modulating an anti-inflammatory response in a patient in need thereof, comprising administering a pharmaceutically acceptable amount of salivaricin peptides to the patient.

According to a certain aspect of the present invention is provided a method of modulating phagocytic activity of neutrophils of a patient in need thereof, comprising administering a pharmaceutically acceptable amount of the pharmaceutical composition or a salivaricin peptide to the patient.

According to a certain aspect of the present invention is provided a method of activating reactive oxygen species of neutrophils in a patient in need thereof, comprising administering a pharmaceutically acceptable amount of the pharmaceutical composition or a salivaricin peptide to the patient.

According to a certain aspect of the present invention is provided a method of activating neutrophil chemotaxis in a patient in need thereof, comprising administering a pharmaceutically acceptable amount of the pharmaceutical composition or a salivaricin peptide to the patient.

According to a certain aspect of the present invention is provided a method of polarizing macrophages towards M2 anti-inflammatory phenotype in a patient in need thereof, comprising administering a pharmaceutically acceptable amount of the pharmaceutical composition or a salivaricin peptide to the patient.

According to a certain aspect of the present invention is provided an isolated S. salivarius strain SALI-10 as deposited as NCBI GeneBank accession number PC090008.

According to a certain aspect of the present invention is provided an isolated S. salivarius strain SALI-10 having a genome sequence of SEQ ID NO: 14.

According to a certain aspect of the present invention is provided an isolated S. salivarius strain SALI-10 having a megaplasmid sequence of SEQ ID NO: 15.

According to a certain aspect of the present invention is provided an isolated S. salivarius strain comprising an SrnM having a sequence of SEQ ID NO:9.

According to a certain aspect of the present invention is provided an isolated S. salivarius strain expressing a peptide SrnA1 having a sequence of SEQ ID NO: 2, a peptide SrnA2 having a sequence of SEQ ID NO: 4, a peptide SrnA4 having a sequence of SEQ ID NO: 8, or post-translationally modified versions thereof.

In certain embodiments, the post translational modifications comprise phosphorylation.

In certain embodiments, the phosphorylation comprises a phosphorylation at Thr4 of each or all of SrnA1, SrnA2 and SrnA4.

In certain embodiments, the post translational modifications comprise a single phosphorylation at Thr4 of each or all of SrnA1, SrnA2 and SrnA4.

In certain embodiments, the post-translational modifications comprise three dehydrations and one phosphorylation.

In certain embodiments, the post-translational modifications consist of three dehydrations and one phosphorylation.

In certain embodiments, the post-translational modifications comprise dehydrations at one or more of amino acids 9, 19 and 21 of each or all of SrnA1, SrnA2 and SrnA4.

In certain embodiments, the post-translational modifications comprise a β-methyllanthionine ring between amino acids 9 and 14 [Abu9-S-Ala14], a β-methyllanthionine ring between amino acids 19 and 24 [Abu19-S-Ala24], and a lanthionine ring between amino acids 21 and 31 [Ala21-S-Ala31], and a phosphorylation at amino acid 4 (Thr4) of each or all of SrnA1, SrnA2 and SrnA4.

### BRIEF DESCRIPTION OF FIGURES

FIG. 1 shows a genetic map of pSALI-10 megaplasmid encoding for the production of salivaricin 10 peptides in strain S. salivarius SALI-10.

FIG. 2 shows the sequence similarity and abundance analysis of the (srn) lanthipeptides system. FIG. 2a: protein similarity map of the lanthionine dehydratase enzyme (SrnM) of strain S. salivarius SALI-10. FIG. 2b: peptide similarity network of the lanthipeptides 4 precursors of the srn operon. FIG. 2c: heatmap for one cluster detected for CGFP analysis of SrnM enzyme using a library of 180 metagenomes from six body sites from healthy individuals.

FIG. 3 summarizes the genetics and proteomics in salivaricin 10 production. FIG. 3a shows a bioactivity assay with the S. salivarius SALI-10 wild-type compared to the delta srn variant. FIG. 3b shows a schematic representation of the srn operon encoding for salivaricin 10 peptides' biosynthesis. FIG. 3c shows the colony mass spectrometry profile of salivaricin 10 producer strain SALI-10 (Black) and plasmid negative variant (gray).

FIG. 4 shows the Structural analysis of Salivaricin 10. FIG. 4a, HR-MS/MS of the SrnA1 (Phenylalanine-variant) peptide containing three dehydrations. FIG. 4b, HR-MS/MS of the SrnA2 (Tyrosine-variant) peptide containing three dehydrations. FIG. 4c, NMR assignment of a threonine at position 4.

FIG. 5 summarizes the bactericidal activity and mode of action of salivaricin 10. FIGS. 5a and 5b shows the killing curve of salivaricin 10. FIG. 5c shows the membrane depolarization activities of nisin A and salivaricin 10 against M. luteus measured using DiOC2(3) fluorescence. FIG. 5d shows Sytox green uptake measurement of membrane permeability activity of salivaricin 10 against M. luteus. FIG. 5e shows the intracellular accumulation of the cell wall precursor UDP-MurNAc-pentapeptide. In FIG. 5f, UDP-MurNAc-pentapeptide was identified by mass spectrometry.

FIG. 6 shows the Antibiofilm activity of salivaricin 10. FIG. 6a, is a schematic of the process. FIG. 6b shows modulation of multispecies biofilm by salivaricin 10 towards commensal-rich microbiome. FIG. 6c shows sum projections of Z-stack images taken by confocal microscopy. A dark black signal indicates viable live cells (Syto 9), a lighter gray signal indicates damaged/dead cells (propidium iodide), viable signal intensity (FIG. 6d) and ratio of viable signal to dead signal (FIG. 6e) were measured by image J. FIG. 6f: The heterogeneity of bacterial distribution (roughness) was measured by viable signal intensity of each pixel using image J. g, Thickness of biofilm was measured after z-stack images from lower part of biofilm to highest part of biofilm (highest signal intensity).

FIG. 7 shows results indicating that Salivaricin 10 augments neutrophils and macrophages priming in human blood and mice bone marrow and induce phagocytosis in vitro. During phagocytosis, salivaricin 10 induced CD markers expression in human neutrophils (FIG. 7a) and macrophages (FIG. 7b). Salivaricin 10 also induced phagocytosis and increased the number of phagocytic cells of neutrophils (FIG. 7c) and macrophages (FIG. 7d). Mice bone marrow neutrophils were also primed by salivaricin 10 with induction of CD markers CD66a, CD55 and CD11b (FIG. 7e) and increase in phagocytosis activity (FIG. 7f).

FIG. 8 shows results of polarization assays. Salivaricin 10 polarizes monocytes towards M2 pro-resolution phenotype. Thp1 monocytes were differentiated with LPS+INF-γ, in the presence or absence of Salivaricin 10 (Sal10). The peptides were found to block M1 polarization, while promoting expression of pro-resolution (M2) macrophage markers (FIG. 8g). In FIG. 8, the gray line shows cells stimulated with IFN and LPS (M1); the black line shows cells stimulated with IFN, LPS and then salivaricin 10 to alter polarization. FIGS. 8a, c, and e show analysis of CD40, CD163 and CD206 respectively. FIGS. 8b, d, f: representative flow spectrum of each CD marker.

FIG. 9 summarizes the findings evidencing that Salivaricin 10 improve phagocytosis in vivo. FIG. 9a, pHrodo E. coli BioParticles conjugates were injected concomitantly with either PBS or salivaricin 10 into the peritoneal cavity. FIG. 9b, percentage of phagocytic neutrophils. FIG. 9c, total signal of phagocytosis in neutrophils.

FIG. 10 shows that Salivaricin 10 induce ROS production by neutrophils and act as neutrophils chemoattractant. FIG. 10a, neutrophils isolated from human blood were exposed to salivaricin 10 at different concentrations. Ros production was induced by salivaricin 10 at a dose dependent manner.

FIG. 10b, a repeated experiment of (a) using neutrophils isolated from bone marrow of mice. FIG. 10c, chemotaxis assay of neutrophils using isolated human neutrophils. Salivaricin 10 concentration at 1 μg produced the highest movement speed.

FIG. 11 shows TOCSY 60 ms NMR Spectrum of a combined HPLC fraction of salivaricin 10 phenylalanine and tyrosine variants in DMSO-d6 recorded at proton frequency of 850 MHz.

FIG. 12 shows NOESY 500 ms NMR Spectrum of a combined HPLC fraction of salivaricin 10 phenylalanine and tyrosine variants in DMSO-d6 recorded at proton frequency of 850 MHz.

FIG. 13 shows the characterization of SrnA4 precursor. FIG. 13a, HR-MS/MS of the SrnA4 peptide. FIG. 13b, SrnA4 mass showing the 2, 3 and 4 charged ions.

FIG. 14 shows a Pairwise Sequence Alignment of srnM lanthionine synthetase of strain SALI10 (SEQ ID NO: 9) and JH (SEQ ID NO: 10), with mutations highlighted.

FIG. 15 shows the immunomodulatory effects of the phosphorylated N-terminal tail of salivaricin 10. FIG. 15a—chemotaxis assay of isolated human neutrophils. Salivaricin 10 was used as chemoattractant and the neutrophil movement speed was monitored. FIG. 15b, Phosphorylated [p(1-8) Sali10], and non-phosphorylated [np(1-8) Sali10] were chemically synthesized and tested in the neutrophil chemotaxis assay at 40 μg/mL. The data shows that phosphate removal resulted in a drastic reduction in neutrophil chemotaxis (p<0.0001), fMLP was used as chemotaxis positive control. FIG. 15c, gene expression data show that phosphorylation is important to downregulate CXCL10 gene associated with M1 macrophages. However, authentic salivaricin 10 showed stronger suppression. FIG. 15d, p(1-8) Sali10 upregulated the gene expression of TGM2 associated with M2 anti-inflammatory macrophages. However, there was no statistically significant difference in the TGM2 gene expression compared with cells treated with the non-phosphorylated analogue. The concentration of peptides used in the gene expression assay was 40 μg/mL. (*)=p<0.05, (**)=p<0.01, (***)=p<0.001. (****)=p<0.0001.

### DETAILED DESCRIPTION

A culture collection of 78 different S. salivarius strains isolated from healthy human adults was screened for the production of inhibitory activities against periodontal disease-associated pathogens and also antibiotic resistant/upper-respiratory tract microorganisms. We found that, while most isolates failed to inhibit P. gingivalis and T. forsythia, extracts from our novel strain S. salivarius SALI-10 were found to have particularly strong capacity to inhibit the growth of these targets. SALI-10 produced the antibacterial substances under nutrient-rich conditions and mostly on semi-solid agar surfaces, and less production was observed in liquid cultures. We have found that SALI-10's novel inhibitory activity is attributed to novel lanthipeptides produced by this strain, encoded on a megaplasmid.

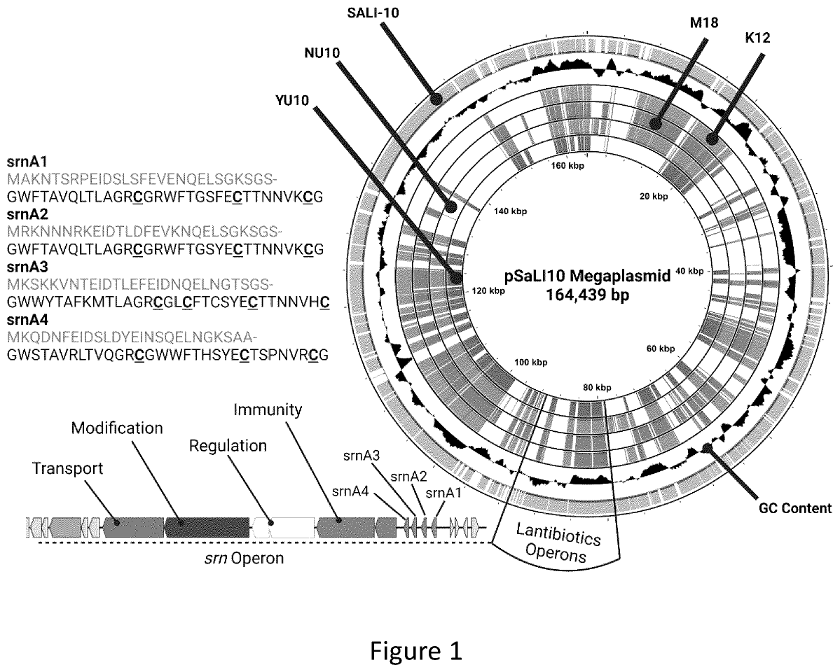

Evidencing this, megaplasmid curing of strain SALI-10 indeed resulted in a new strain SALI-10-PSaLI10 lacking the characteristic inhibitory activity. Whole genome sequencing on S. salivarius SALI-10 revealed the presence of one lanthipeptide encoding operon (srn) harbored on a 164 kb megaplasmid, submitted as NCBI GenBank accession number CP090008 (incorporated herein by reference). The complete DNA sequence of the SALI-10 genome is shown as SEQ ID NO:14. The complete DNA sequence of the SALI-10 megaplasmid is shown as SEQ ID NO:15. FIG. 1 shows the genetic map of pSALI-10 megaplasmid encoding for the production of salivaricin 10 peptides in strain S. salivarius SALI-10. BLAST was performed against other megaplasmids encoding other lantibiotics salivaricins production in S. salivarius strains K12, M18, NU10 and YU10. The srn operon responsible for the production of the lanthipeptides is shown and include characteristic class II LanM dehydratase, response regulator and histidine kinase, putative self-immunity genes and ABC transporter. Salivaricin 10 peptides precursor translated in silico are shown in the top left of FIG. 1, the gray sequence is the leader peptide, and the black sequence is

(srnA1 full peptide SEQ ID NO: 1:
MAKNTSRPEIDSLSFEVENQELSGKSGSGWFTAVQLTLAGRCGRWFTGSFECTTNNVKCG; srnA1 mature peptide
SEQ ID NO: 2: GWFTAVQLTLAGRCGRWFTGSFECTTNNVKCG; srnA2 full peptide SEQ ID NO: 3:
MRKNNNRKEIDTLDFEVKNQELSGKSGSGWFTAVQLTLAGRCGRWFTGSYECTTNNVKCG; srnA2 mature
peptide SEQ ID NO: 4: GWFTAVQLTLAGRCGRWFTGSYECTTNNVKCG; srnA3 full peptide SEQ ID NO: 5:
MKSKKVNTEIDTLEFEIDNQELNGTSGSGWWYTAFKMTLAGRCGLCFTCSYECTTNNVHC; srnA3 mature peptide
SEQ ID NO: 6: GWWYTAFKMTLAGRCGLCFTCSYECTTNNVHC; srnA4 full peptide SEQ ID NO: 7:
MKQDNFEIDSLDYEINSQELNGKSAAGWSTAVRLTVQGRCGWWFTHSYECTSPNVRCG; srnA4 mature peptide
SEQ ID NO: 8: GWSTAVRLTVQGRCGWWFTHSYECTSPNVRCG).

As shown in FIG. 1, the srn operon in strain SALI-10 harbored all necessary genes for the production of 4 ribosomally synthesized and posttranslationally modified peptides.

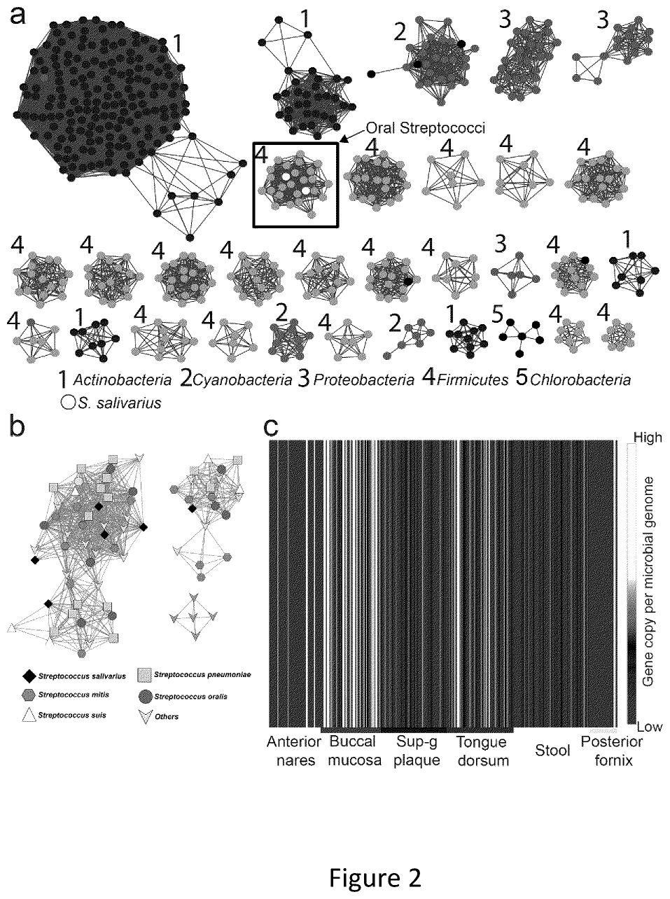

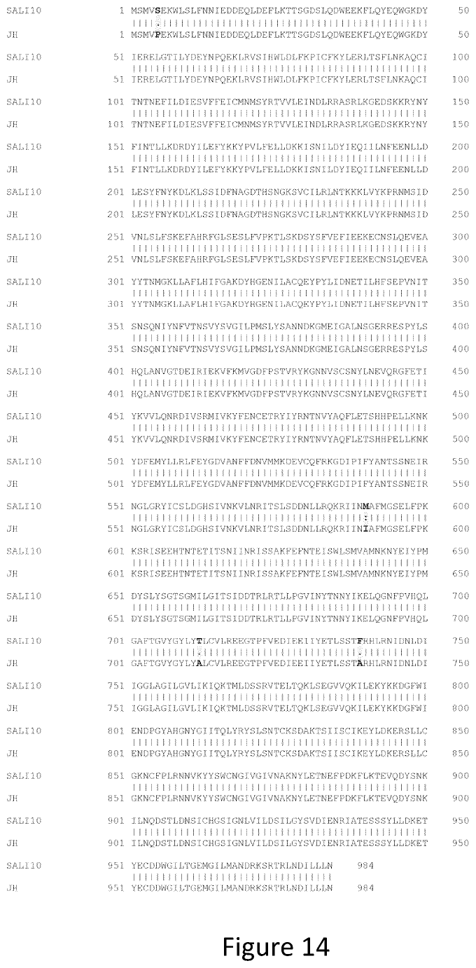

We have found that the lanthionine synthetase enzyme is an important component of the post-translation modification process to produce the bioactive polycyclic lanthipeptides. To determine the phylogenetic distribution of SrnM enzyme, a sequence similarity network was constructed with the Enzyme Function Initiative Enzyme Similarity Tool (EFI-EST)[21] utilizing all members of the lanthionine synthetase family from the UniProtKB database. The relative similarity between individual enzymes was performed with Cytoscape[22] at an alignment score threshold of 130. Actinobacteria constitute the majority of the enzyme sequences found in the UniProtKB database, with one primary cluster and another smaller one. The analysis also identified one Cyanobacteria cluster and two Proteobacteria clusters. Multiple small-size Firmicutes clusters resulted from this analysis, with one specific group containing only oral streptococci, including the species S. salivarius, S. mitis, S. oralis and S. pneumoniae. This separate cluster showed no close linkage to any other group and contained strains encoding for SrnM enzyme, suggesting that SrnM is specifically attributed to lanthipeptides synthesized in the oral cavity (FIG. 2-a). Four mutations within the srnM gene were identified in strain SALI-10 compared to that reported for salivaricin E in strain JH (FIG. 14; SrnM of SALI10 is shown as SEQ ID NO:9; SrnM of JH is shown as SEQ ID NO: 10), which can results in different posttranslation modifications. Similar approach was applied on the SrnA peptide precursors. Database search on SrnA1, SrnA2, SrnA3 and SrnA4 at an identification cut-off of 65% also showed that these peptides were unique to the oral streptococci group (FIG. 2-b). We used chemically guided functional profiling (CGFP) to map SrnM enzyme abundance in a library of 180 different metagenomes from different body sites using sequence similarity networks generated by the EFI-EST web tool. Abundance quantitation of SrnM enzyme among metagenomic databases revealed that this enzyme is primarily found on the buccal mucosa, tongue dorsum and supragingival plaque, respectively (FIG. 2-c). Other body sites including the gastrointestinal tract and anterior nares showed less hits suggesting that this enzyme and its operon are unique to bacteria colonizing the oral cavity. Biosynthetic gene clusters encoding for secondary metabolites production are conserved in most Actinobacteria genera including Streptomyces but are relatively rare in the human microbiome. The srn operon was almost exclusively found in oral streptococcal strains and it encodes for the production of a combination of lanthipeptides modified by one class II lanthionine dehydratase enzyme (LanM).

FIG. 2 shows the sequence similarity and abundance analysis of the (srn) lanthipeptides system. FIG. 2a shows the protein similarity map of the lanthionine dehydratase enzyme (SrnM) of strain S. salivarius SALI-10. Each node represents one protein sequence, each edge represents BLASTP E-values cut-off of 1E-130. FIG. 2b shows the peptide similarity network of the lanthipeptides 4 precursors of the srn operon. Each node represents one peptide sequence, each edge represents BLASTP with similarity cut of 65%. The sequence similarity networks were generated using the Enzyme Similarity Tool-Enzyme Function Initiative (EST-EFI, https://efi.igb.illinois.edu/efi-est/) and visualized in Cytoscape (v.3.9.0). FIG. 2c shows the heatmap for one cluster detected for CGFP analysis of SrnM enzyme using a library of 180 metagenomes from six body sites from healthy individuals. The detected cluster is displayed on the y-axis, the body sites on the x-axis. The bar on the right defines the color key for the “gene copies per microbial genome”.

### Salivaricin 10: A Novel Combination of Phosphorylated Lanthipeptides

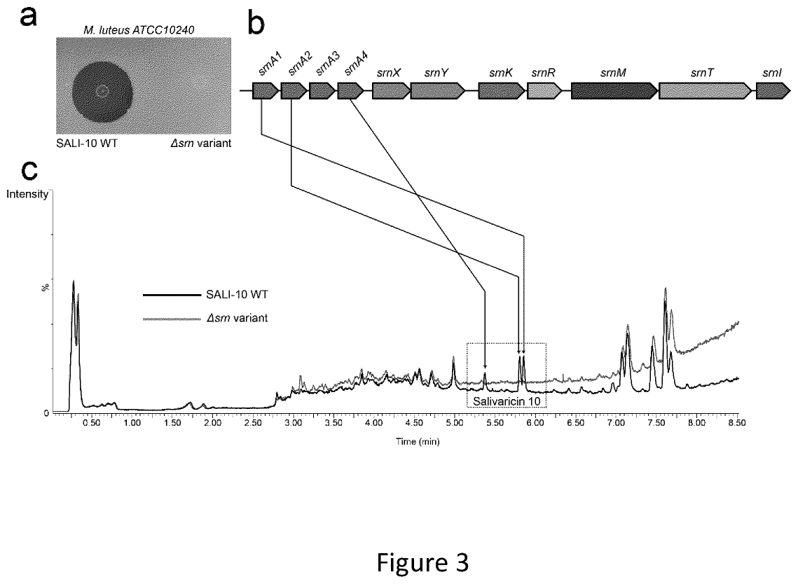

The antibacterial activity of the producer strain S. salivarius SALI-10 was enriched by isopropanol extraction of cells grown on agar plates. To access milligram quantities of each compound, the biosynthesis was induced by supplementing agar cultures of the strain S. salivarius SALI-10 with crude extracts prepared from previous uninduced cultures of the same producer. A megaplasmid-cured mutant was generated from the SALI-10 strain, which was shown to have lost antimicrobial activity (FIG. 3-a). Reverse phase ultra-performance liquid chromatography equipped with positive electrospray ionization time-of-flight mass spectrometry (RP-UPLC-ES+-TOF-MS) revealed obvious differences between mass spectra of cell extracts from SALI-10 wild-type and megaplasmid-cured mutant (SALI-10^{-PSaLI10}). As demarcated in FIG. 3-c, three products were not present in the cured mutant.

FIG. 3 summarizes the genetics and proteomics in salivaricin 10 production. FIG. 3a shows a bioactivity assay with the S. salivarius SALI-10 wild-type, the Δsrn variant strain (−PSaLI10) was tested as a negative control, and it lacks srn locus responsible for producing salivaricin 10 peptides. Both SALI-10 and Δsrn variant strains were spotted on the surface of Tryptic Soy Agar plates and grew for 18 hours before overlaying with a sensitive bacterial target. Inhibition zone of no target bacteria growth is observed as a result of the antibacterial action of salivaricin 10. Salivaricin 10 deficient strain could not inhibit the target bacteria. FIG. 3b shows a schematic representation of the srn operon encoding for salivaricin 10 peptides' biosynthesis. The operon contains genes represent 4 peptide precursors to be ribosomally synthesized, genes with putative self-immunity role, genes involved in histidine kinase regulation and sensitizing, a gene encodes the lanthipeptides modification enzyme, a gene encodes lanthipeptide transporter and a gene with likely a peptide isomerase function. FIG. 3c shows the colony mass spectrometry profile of salivaricin 10 producer strain SALI-10 (black) and plasmid negative variant (gray). Cell extracts were compared by reversed-phase UPLC-TOF-MS-ES+. The three peptides composing salivaricin 10 are indicated in a dashed box.

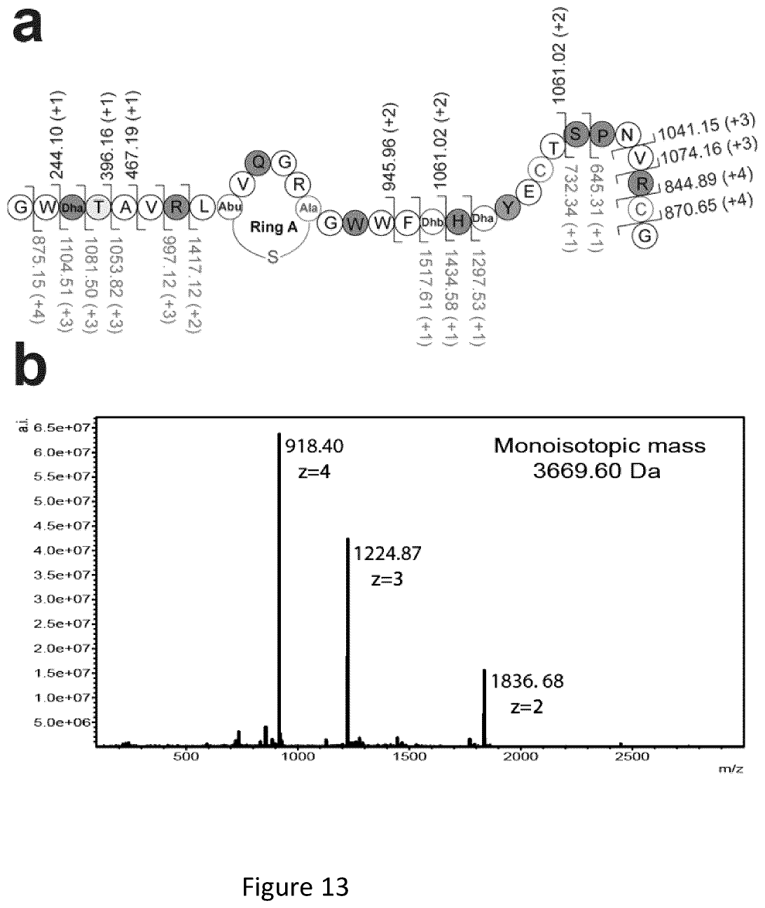

The two antimicrobial products eluting between 5.8 and 6.0 minutes (FIG. 3-c) were found to have a monoisotopic mass determined by high-resolution, accurate-mass spectrometry (HRMS) of 3551.58 Da and 3535.58 Da, respectively. The masses differed by the presence of a tyrosine or phenylalanine at position 22 in the core peptide sequence. The less-inhibitory peptide eluting at 5.3 minutes (FIG. 3-c) showed a monoisotopic mass of 3669.60 Da (FIG. 13).

Interestingly, the peptides were larger than the predicted mass according to the peptide sequence. High-resolution mass spectrometry (HRMS) on the two most inhibitory peptides (SrnA1 and SrnA2) revealed that the products were all phosphorylated, and further non-phosphorylated variants were not observed (Tables 1, 2, 3, 4). The data also showed that the peptides did not undergo more than three dehydrations and that the peptides contained primarily two or three dehydrations (Tables 2 and 4). The chemical formula for each peptide containing three dehydrations was found to be C₁₅₅H₂₂₇N₄₄O₄₄S₃P₁ (SrnA1 (phe-variant); Theoretical ([M+3H]/3)³⁺: m/z 1,179.53; Observed ([M+3H]/3)³⁺: m/z 1,179.53; mass error: −0.61 [p.p.m]), C₁₅₅H₂₂₇N₄₄O₄₅S₃P₁ (SrnA2 (tyr-variant); Theoretical ([M+3H]/3)³⁺: m/z 1,184.86; Observed ([M+3H]/3)³⁺: m/z 1,184.86; mass error: 1.78 [p.p.m.]), C₁₆₀H₂₂₉N₄₈O₄₅S₃P₁ (SrnA4); Theoretical ([M+4H]/4)⁴⁺: m/z 918.40; Observed ([M+4H]/4)⁴⁺: m/z 918.40; mass error: −2.43 [p.p.m]) The molecular formulas and masses of all three peptides did not correspond to any known salivaricin structures.

TABLE 1
Salivaricin 10 non-phosphorylated phenylalanine-variant. No mass ions
were detected for such a variant containing zero, one, two, three
of four dehydrations are present in the isolated HPLC fraction.

| | | | | m/z | m/z | m/z |
|---|---|---|---|---|---|---|
| Dehydration | Phosphate | Charge | Area | (Expected) | (Observed) | (ppm) |
| 0 | 0 | [M + 1H] | 0 | 3510.6504 | 0 | 0 |
| 0 | 0 | [M + 2H] | 0 | 1755.8288 | 0 | 0 |
| 0 | 0 | [M + 3H] | 0 | 1170.8883 | 0 | 0 |
| 0 | 0 | [M + 4H] | 0 | 878.4181 | 0 | 0 |
| 1 | 0 | [M + 1H] | 0 | 3492.6398 | 0 | 0 |
| 1 | 0 | [M + 2H] | 0 | 1746.8236 | 0 | 0 |
| 1 | 0 | [M + 3H] | 0 | 1164.8848 | 0 | 0 |
| 1 | 0 | [M + 4H] | 0 | 873.9154 | 0 | 0 |
| 2 | 0 | [M + 1H] | 0 | 3474.6293 | 0 | 0 |
| 2 | 0 | [M + 2H] | 0 | 1737.8183 | 0 | 0 |
| 2 | 0 | [M + 3H] | 0 | 1158.8813 | 0 | 0 |
| 2 | 0 | [M + 4H] | 0 | 869.4128 | 0 | 0 |
| 3 | 0 | [M + 1H] | 0 | 3456.6187 | 0 | 0 |
| 3 | 0 | [M + 2H] | 0 | 1728.813 | 0 | 0 |
| 3 | 0 | [M + 3H] | 0 | 1152.8778 | 0 | 0 |
| 3 | 0 | [M + 4H] | 0 | 864.9101 | 0 | 0 |
| 4 | 0 | [M + 1H] | 0 | 3438.6081 | 0 | 0 |
| 4 | 0 | [M + 2H] | 0 | 1719.8077 | 0 | 0 |
| 4 | 0 | [M + 3H] | 0 | 1146.8742 | 0 | 0 |
| 4 | 0 | [M + 4H] | 0 | 860.4075 | 0 | 0 |

TABLE 2
Salivaricin 10 phosphorylated phenylalanine-variant. M + 3H
and M + 4H charged ions with the expected masses were detected
for a phosphorylated product containing one, two, or three dehydrations
(primarily two and three dehydration based on area counts). Observed
masses are within 10 ppm of expected mass.

| | | | | m/z | m/z | m/z |
|---|---|---|---|---|---|---|
| Dehydration | Phosphate | Charge | Area | (Expected) | (Observed) | (ppm) |
| 0 | 1 | [M + 1H] | 0 | 3590.6167 | 0 | 0 |
| 0 | 1 | [M + 2H] | 0 | 1795.812 | 0 | 0 |
| 0 | 1 | [M + 3H] | 0 | 1197.5438 | 0 | 0 |
| 0 | 1 | [M + 4H] | 0 | 898.4096 | 0 | 0 |
| 1 | 1 | [M + 1H] | 0 | 3572.6062 | 0 | 0 |
| 1 | 1 | [M + 2H] | 0 | 1786.8067 | 0 | 0 |
| 1 | 1 | [M + 3H] | 7176560 | 1191.5402 | 1191.537 | −2.7298 |
| 1 | 1 | [M + 4H] | 5287632 | 893.907 | 893.9048 | −2.4777 |
| 2 | 1 | [M + 1H] | 0 | 3554.5956 | 0 | 0 |
| 2 | 1 | [M + 2H] | 0 | 1777.8014 | 0 | 0 |
| 2 | 1 | [M + 3H] | 49422948 | 1185.5367 | 1185.5323 | −3.6872 |
| 2 | 1 | [M + 4H] | 32836691 | 889.4044 | 889.4015 | −3.2277 |
| 3 | 1 | [M + 1H] | 0 | 3536.585 | 0 | 0 |
| 3 | 1 | [M + 2H] | 0 | 1768.7962 | 0 | 0 |
| 3 | 1 | [M + 3H] | 18672655 | 1179.5332 | 1179.5325 | −.6098 |
| 3 | 1 | [M + 4H] | 11700963 | 884.9017 | 884.9017 | .0151 |
| 4 | 1 | [M + 1H] | 0 | 3518.5745 | 0 | 0 |
| 4 | 1 | [M + 2H] | 0 | 1759.7909 | 0 | 0 |
| 4 | 1 | [M + 3H] | 0 | 1173.5297 | 0 | 0 |
| 4 | 1 | [M + 4H] | 0 | 880.3991 | 0 | 0 |

TABLE 3
Salivaricin 10 non-phosphorylated tyrosine-variant. No ions with the expected
masses were detected for such a variant containing zero, one, two, three
of four dehydrations are present in the isolated HPLC fraction.

| | | | | m/z | m/z | m/z |
|---|---|---|---|---|---|---|
| Dehydration | Phosphate | Charge | Area | (Expected) | (Observed) | (ppm) |
| 0 | 0 | [M + 1H] | 0 | 3526.6453 | 0 | 0 |
| 0 | 0 | [M + 2H] | 0 | 1763.8263 | 0 | 0 |
| 0 | 0 | [M + 3H] | 0 | 1176.22 | 0 | 0 |
| 0 | 0 | [M + 4H] | 0 | 882.4168 | 0 | 0 |
| 1 | 0 | [M + 1H] | 0 | 3508.6347 | 0 | 0 |
| 1 | 0 | [M + 2H] | 0 | 1754.821 | 0 | 0 |
| 1 | 0 | [M + 3H] | 0 | 1170.2164 | 0 | 0 |
| 1 | 0 | [M + 4H] | 0 | 877.9141 | 0 | 0 |
| 2 | 0 | [M + 1H] | 0 | 3490.6242 | 0 | 0 |
| 2 | 0 | [M + 2H] | 0 | 1745.8157 | 0 | 0 |
| 2 | 0 | [M + 3H] | 0 | 1164.2129 | 0 | 0 |
| 2 | 0 | [M + 4H] | 0 | 873.4115 | 0 | 0 |
| 3 | 0 | [M + 1H] | 0 | 3472.6136 | 0 | 0 |
| 3 | 0 | [M + 2H] | 0 | 1736.8105 | 0 | 0 |
| 3 | 0 | [M + 3H] | 0 | 1158.2094 | 0 | 0 |
| 3 | 0 | [M + 4H] | 0 | 868.9089 | 0 | 0 |
| 4 | 0 | [M + 1H] | 0 | 3454.6031 | 0 | 0 |
| 4 | 0 | [M + 2H] | 0 | 1727.8052 | 0 | 0 |
| 4 | 0 | [M + 3H] | 0 | 1152.2059 | 0 | 0 |
| 4 | 0 | [M + 4H] | 0 | 864.4062 | 0 | 0 |

TABLE 4
Salivaricin 10 phosphorylated tyrosine-variant. M + 3H and M + 4H
charged ions with the expected masses were detected for a phosphorylated product
containing one, two, or three dehydrations (primarily two and three dehydration
based on area counts). Observed masses are within 10 ppm of expected mass.

| | | | | m/z | m/z | m/z |
|---|---|---|---|---|---|---|
| Dehydration | Phosphate | Charge | Area | (Expected) | (Observed) | (ppm) |
| 0 | 1 | [M + 1H] | 0 | 3606.6116 | 0 | 0 |
| 0 | 1 | [M + 2H] | 0 | 1803.8095 | 0 | 0 |
| 0 | 1 | [M + 3H] | 0 | 1202.8754 | 0 | 0 |
| 0 | 1 | [M + 4H] | 0 | 902.4084 | 0 | 0 |
| 1 | 1 | [M + 1H] | 0 | 3588.6011 | 0 | 0 |
| 1 | 1 | [M + 2H] | 0 | 1794.8042 | 0 | 0 |
| 1 | 1 | [M + 3H] | 1514273 | 1196.8719 | 1196.8679 | −3.3087 |
| 1 | 1 | [M + 4H] | 825589 | 897.9057 | 897.905 | −.8483 |
| 2 | 1 | [M + 1H] | 0 | 3570.5905 | 0 | 0 |
| 2 | 1 | [M + 2H] | 0 | 1785.7989 | 0 | 0 |
| 2 | 1 | [M + 3H] | 23771360 | 1190.8684 | 1190.8716 | 2.7140 |
| 2 | 1 | [M + 4H] | 16384948 | 893.4031 | 893.4056 | 2.7967 |
| 3 | 1 | [M + 1H] | 0 | 3552.58 | 0 | 0 |
| 3 | 1 | [M + 2H] | 0 | 1776.7936 | 0 | 0 |
| 3 | 1 | [M + 3H] | 32714476 | 1184.8648 | 1184.8669 | 1.7836 |
| 3 | 1 | [M + 4H] | 21941586 | 888.9004 | 888.9026 | 2.4850 |
| 4 | 1 | [M + 1H] | 0 | 3534.5694 | 0 | 0 |
| 4 | 1 | [M + 2H] | 0 | 1767.7883 | 0 | 0 |
| 4 | 1 | [M + 3H] | 0 | 1178.8613 | 0 | 0 |
| 4 | 1 | [M + 4H] | 0 | 884.3978 | 0 | 0 |

To access milligram quantities of the peptides, the biosynthesis was induced by supplementing agar cultures of the strain S. salivarius SALI-10 with nanomolar quantities of crude extracts prepared from previous uninduced cultures of the same producer. Cultivation of the induced cultures in semi-solid media led to the production of strong inhibitory activity which could be purified by consecutive steps of chloroform extraction, C18 solid phase extraction, and C18 reversed phase HPLC. The pure compounds exhibited exactly the same masses as determined for the compounds from the wild-type strain SALI-10 and was named salivaricin 10.

High-resolution, accurate-mass tandem mass spectrometry (MS/MS) was performed to identify the region of the peptide that was phosphorylated. Given that all peptide variants were phosphorylated, the threonine residues at positions 4, 25, and 26 were considered as likely sites for phosphorylation. MS/MS on the ([M+3H]/3)³⁺ ions observed in the HRMS data for the SrnA1 and SrnA2 products containing three dehydrations and were analyzed to determine the site/s of phosphorylation. The higher energy collisional dissociation (HCD)-based fragmentation of these ions is shown in Error! Reference source not found.-a,b. The fragment b and y ions provided evidence for Thr4 to be the phosphorylation site on the SrnA1 (phe-variant) and SrnA2 (tyr-variant) peptides. Assigned b ions between m/z 474.21 and m/z 772.39 had masses corresponding to a single dehydration, while the masses m/z 1140.86 (SrnA1, Phe variant) and m/z 1146.20 (SrnA2, Tyr variant) correspond to four dehydrations. The assigned y ion measuring m/z 1773.72 (SrnA2, Tyr variant) corresponds to two dehydrations. The y ions between m/z 1277.58-1483.21 (phe-variant) and the m/z 1342.12-1532.73 (tyr-variant) correspond to three dehydrations, while the y ions between m/z 1524.72-1146.87 (phe-variant) and the m/z 1606.26-1152.21 (tyr-variant) correspond to four dehydrations. Given that the ion used for MS/MS had only three dehydrations and a phosphate, the observation of an additional dehydration was due to the loss of phosphate. When the phosphate dissociates during fragmentation, the fragment is observed as a dehydrated product. In all cases, there were no additional ions to support the presence of the phosphate in any other location within the peptide, such as Thr25 and Thr26. The proposed lanthionine ring formations in SrnA1 (Phe-variant) and SrnA2 (Try-variant) occur between a dehydrated threonine (Dhb) at position 9 and a cysteine at position 14. The masses observed and the HCD-induced MS/MS fragmentation pattern for both the SrnA1 (Phe-variant) and SrnA2 (Tyr-variant) support the proposed assignment of the lanthionine rings. There were no observable fragment ions, within the acceptable limits, within the regions spanned by the proposed thioether linkages. Two-dimensional NMR analyses confirmed that Thr5 is not a dehydrated Dhb residue, supporting that the dehydration is due to the loss of phosphate in the MS/MS experiments (Error! Reference source not found.-c). Nuclear Overhauser effects (NOEs) provided sequential walk assignments (i+1) for the N-terminal linear peptide region of the SrnA1 (Phe-variant) and SrnA2 (Tyr-variant) products. The proton chemical shift values for Gly1-Trp2-Phe3-Thr4-Ala5-Val6-Gln7-Leu8 were provided in Table 5, below. The assigned spin system for Thr4 (8.18 ppm [NH], 4.34 ppm [Hα], 4.14 ppm [Hβ], and 1.05 ppm [Hγ]) was clearly indicative that it is not a dehydrated Dhb residue and the observed loss of water in the MS/MS data at this position is therefore due to the loss of the phosphate.

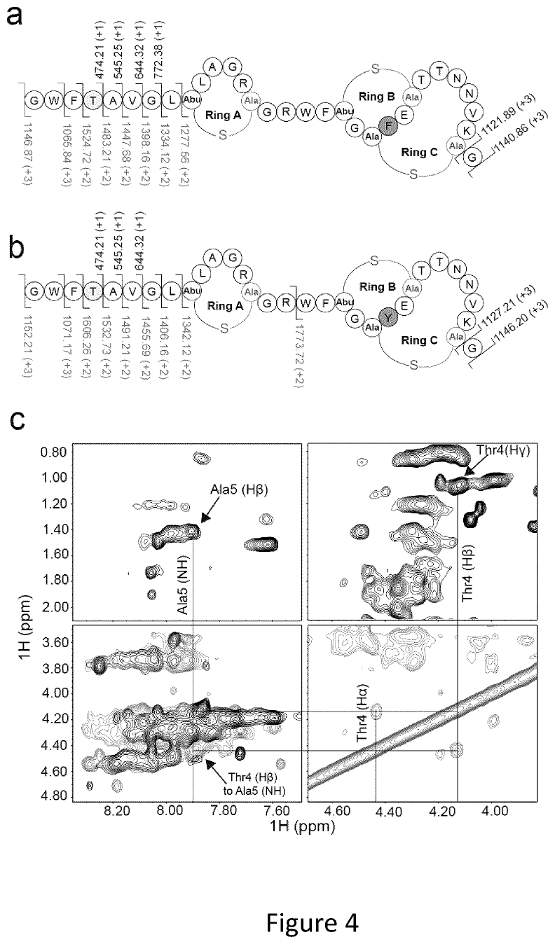

FIG. 4 shows the Structural analysis of Salivaricin 10. a, HR-MS/MS of the SrnA1 (Phenylalanine-variant) peptide containing three dehydrations. b, HR-MS/MS of the SrnA2 (Tyrosine-variant) peptide containing three dehydrations. The ions with a signal/noise (s/n) greater than 5.0 were used for assignments and all but one ion for each peptide had less than 10 ppm mass difference from expected and observed masses (Tables 6 and 7). The masses m/z 1121.89 (SrnA1, Phe variant) and m/z 1127.21 (SrnA2, Tyr variant) had the correct isotopic ratios for the corresponding peptides and the C13 isotopes were within the acceptable 10 ppm limits. b and y ions are labeled above and below amino acids respectively. c, NMR assignment of a threonine at position 4. A sequential walk from the alpha and beta protons on the phosphorylated threonine to the amide spine system of alanine at position 5. The alpha and beta protons for Thr4 are shifted downfield due to the presence of a phosphate. NOESY spectra and TOCSY spectra are show in gray and black, respectively.

TABLE 5
Proton Chemical Shift Values for Salivaricin
10 N-terminal linear peptide (Gly1 to Leu8).

| Amino | | | | |
|---|---|---|---|---|
| Acid | HN | Hα | Hβ | Other |
| Gly1 | n.d. | 4.24 | | |
| Trp2 | 8.04 | 4.55 | 3.07, 2.89 | Hδ1 7.08, Hε1 10.86, Hε3 7.31, |
| | | | | Hζ2 7.21 Hζ3 6.68 |
| Phe3 | 8.29 | 4.71 | 2.99, 2.86 | Hδ1, Hδ2, Hε1, Hε2, Hζ1 7.24 |
| Thr4 | 8.18 | 4.44 | 4.13 | Hγ2 1.05 |
| Ala5 | 7.89 | 4.31 | 1.41 | |
| Val6 | 8.05 | 4.26 | 1.90, 1.73 | Hγ 0.84 |
| Gln7 | 7.98 | 4.49 | 2.12 | Hγ2 and Hγ3 n.d., Hε21 7.21, |
| | | | | Hε22 6.9 |
| Leu8 | 7.98 | n.d. | 1.42 | Hγ n.d, Hδ 0.87 |

n.d. Values were not detected or were not assignable due to peak overlap

TABLE 6
Fragment ions of SrnA1 peptide.

| b ions | | | y ions | | |
|---|---|---|---|---|---|
| Observed | Charge | Δm/z (ppm) | Observed | Charge | Δm/z (ppm) |
| 474.21 | +1 | −6.65 | 1277.58 | +2 | −6.25 |
| 545.25 | +1 | −10.12 | 1334.12 | +2 | −4.76 |
| 644.32 | +1 | −9.39 | 1398.16 | +2 | −7.70 |
| 772.38 | +1 | −4.14 | 1447.68 | +2 | −2.21 |
| 1121.89 | +3 | −24.61 | 1483.21 | +2 | −8.10 |
| 1140.86 | +3 | −3.45 | 1524.72 | +2 | −5.78 |
| | | | 1065.84 | +3 | −9.05 |
| | | | 1146.87 | +3 | −7.02 |

TABLE 7
Fragment ions of SrnA2 peptide.

| b ions | | | y ions | | |
|---|---|---|---|---|---|
| Observed | Charge | Δm/z (ppm) | Observed | Charge | Δm/z (ppm) |
| 474.21 | +1 | −3.94 | 1773.72 | +1 | 3.18 |
| 545.25 | +1 | −5.10 | 1342.12 | +2 | −10.46 |
| 644.32 | +1 | −3.35 | 1406.16 | +2 | −9.25 |
| 1127.21 | +3 | 18.66 | 1455.69 | +2 | −5.95 |
| 1146.20 | +3 | −7.39 | 1491.21 | +2 | −8.35 |
| | | | 1532.73 | +2 | −9.54 |
| | | | 1606.26 | +2 | −7.62 |
| | | | 1071.17 | +3 | −3.64 |
| | | | 1152.21 | +3 | −7.97 |

TABLE 8
Fragment ions of SrnA4 peptide.

| b ions | | | y ions | | |
|---|---|---|---|---|---|
| | | Δm/z | | | Δm/z |
| Observed | Charge | (ppm) | Observed | Charge | (ppm) |
| 244.10 (b2) | +1 | −0.81 | 645.30 (y6) | +1 | −6.19 |
| 396.16 (b4) | +1 | 0.25 | 732.34 (y7) | +1 | −4.36 |
| 467.20 (b5) | +1 | −6.20 | 1297.53 (y12) | +1 | 1.46 |
| 945.96 (b17) | +2 | −2.43 | 717.80 (y13) | +2 | 3.76 |
| 1061.02 (b19) | +2 | 4.42 | 1517.61 (y14) | +1 | −7.70 |
| 941.77 (b25) | +3 | 8.22 | 925.89 (y16) | +2 | 3.34 |
| 1041.15 (b28) | +3 | 9.43 | 907.40 (y24) | +3 | 10.35 |
| 1074.16 (b29) | +3 | 4.93 | 1417.12 (y25) | +2 | −2.62 |
| 844.89 (b30) | +4 | −11.24 | 997.12 (y26) | +3 | 5.51 |
| 870.65 (b31) | +4 | 2.87 | 1053.82 (y28) | +3 | 3.79 |
| | | | 1081.50 (y29) | +3 | 0.92 |
| | | | 1104.51 (y30) | +3 | 4.16 |
| | | | 875.15 (y31) | +4 | 0.10 |

### Salivaricin 10 Activity Against Major Oral Pathogens and Primary Mode of Action

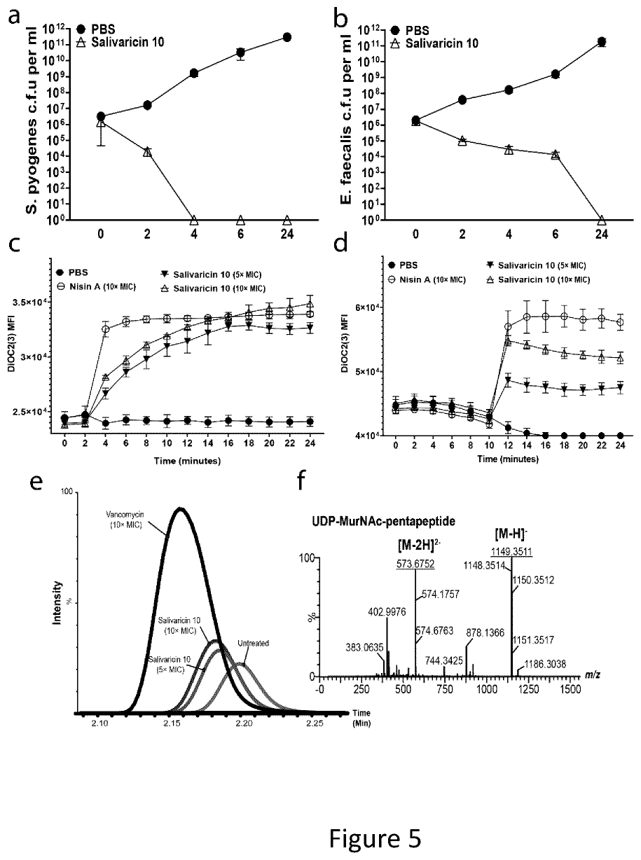

Salivaricin 10 was found to have a potent antimicrobial activity against a wide range of Gram-positive bacteria, including opportunistic pathogen such as MDR Streptococcus pneumonia, Streptococcus pyogenes, Streptococcus dysagalactiae, vancomycin-resistant Enterococcus faecium and multi-drug resistant Enterococcus faecalis clinical isolates. Interestingly, the spectrum of antibacterial activity of salivaricin 10 includes a number of Gram-negative diseases-associated pathogens including P. gingivalis, T. forsythia and Neisseria gonorrhea. Minimal inhibitory concentration (MIC) values of salivaricin 10 were found to be in the micromolar range (0.125-64 μg ml⁻¹, for Gram-positive bacteria) and (32-64 μg ml⁻¹, for Gram-negative bacteria) demonstrating high potency (show in Table 9, below). Salivaricin 10 was bactericidal against targeted bacteria including those tested in time killing assay like S. pyogenes and E. faecalis. E. faecalis was tested here as one of the main targets of salivaricin 10 since this bacterium contains membrane vesicles (MVs) that possess unique lipid and protein profiles, distinct from the intact cell membrane and are enriched in lipoproteins. E. faecalis MVs contribute to antimicrobial resistance and host immune evasion[23]. Salivaricin 10 rapidly reduced the number of E. faecalis cells upon exposure of 2 hours. The killing kinetics was logarithmic after 2, 4 and 6 hours of exposure. Total killing of E. faecalis cells was observed after 24 hours of exposure. S. pyogenes cells, on the other hand were completely killed after 4 hours of exposure without any cells re-grown after 24 hours of exposure. This was clear evidence of the bactericidal activity of salivaricin 10, as summarized in FIG. 5, which summarizes the bactericidal activity and mode of action of salivaricin 10. FIGS. 5a and 5b showed the killing curve of salivaricin 10, as follows. Incubation of either E. faecalis or S. pyogenes with a 10×MIC of salivaricin 10 led to complete killing of the inoculum after 24 h. Data represent medians±s.d. of three independent experiments. FIG. 5c shows the membrane depolarization activities of nisin A and salivaricin 10 against M. luteus measured using DiOC2(3) fluorescence.

Antimicrobials were added after 2 minutes. FIG. 5d shows Sytox green uptake measurement of membrane permeability activity of salivaricin 10 against M. luteus. Antimicrobials were added after 10 minutes. FIG. 5e shows the intracellular accumulation of the cell wall precursor UDP-MurNAc-pentapeptide. Whole cells of M. luteus were treated with salivaricin 10 at 5× or 10×MIC. Untreated and vancomycin treated (10×MIC) cells were used as controls. UPLC-MS analyzed the accumulated cell wall precursor on negative mode, retention times shift due to the order of magnitude changes in concentration. In FIG. 5f, UDP-MurNAc-pentapeptide was identified by mass spectrometry as indicated by the peaks at m/z 1,149.3511 ([M−H]− ion) and m/z 573.6752 ([M−2H]²⁻ ion).

TABLE 9
Activity of salivaricin 10 against pathogenic microorganisms.

| | | Antibiotic | Salivaricin |
|---|---|---|---|
| Organism | Strain | resistance profile | 10 MIC (μg/mL) |
| M. luteus | ATCC10240 | NA | 0.125 |
| S. pyogenes | W51503011 | CLI-ERY (R) | 16 |
| S. pyogenes | W80908630 | CLI-ERY (S) | 16 |
| S. agalactiae | W73106655 | CLI-ERY (R) | 16 |
| S. agalactiae | W70407881 | CLI-ERY (S) | 32 |
| S. | V4122578 | CRO-CLI-DOX-ERY- | 0.5 |
| pneumoniae | | LVX-MXF (R) | |
| S. | W81002644 | OXA-PENIV-SXT (R) | 1 |
| pneumoniae | | | |
| E. faecalis | W82000259 | QD-TET-ERY (R) | 32 |
| E. faecalis | W80909324 | QD-TET-ERY (S) | 32 |
| E. faecalis | ATCC29212 | NA | 16 |
| E. feacium | W97542221 | VAN (R) | 64 |
| S. mutans | UA159 | NA | >64 |
| N. | V3091967 | CIP (R) | 64 |
| gonorrhoeae | | | |
| P. gingivalis | ATCC33277 | NA | 16 |
| T. forsynthia | ATCC43037 | NA | 32 |

(R): resistant,
(S): sensitive,
NA: not available.
CLI: clindamycin,
ERY: erythromycin,
CRO: ceftriaxone,
DOX: doxycycline,
LVX: levofloxacin,
MXF: moxifloxacin,
OXA: oxacillin,
PENIV: penicillin
V, SXT: Trimethoprim/Sulfamethoxazole,
QD: quinupristin/dalfopristin,
TET: tetracycline,
VAN: vancomycin,
CIP: ciprofloxacin.

### Activity Against Saliva-Derived Multispecies Biofilms

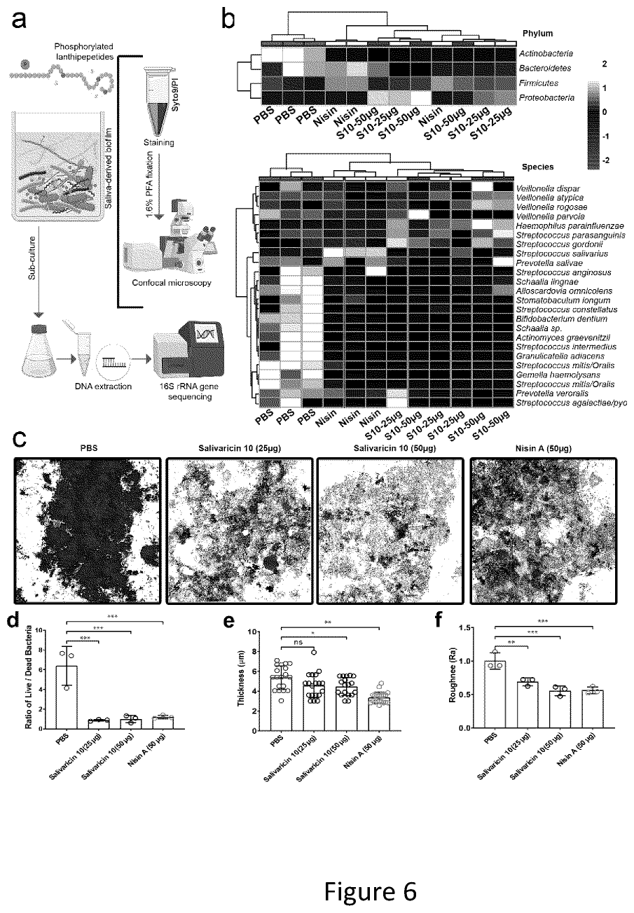

The bioactivity of salivaricin 10 against saliva-derived multispecies biofilms was established using both 16S rRNA gene sequencing and confocal microscopy. Salivaricin 10 improved total Firmicutes and Proteobacteria numbers in the multispecies biofilm while suppressing Actinobacteria and Bacteroidetes (FIG. 6-b). Salivaricin 10 treatment diminished pulmonary abscess-associated bacteria, Actinomyces graevenitzii and Granulicatella adiacens, and the opportunistic cariogen Bifidobacterium dentium. Species related to the genus Prevotella were also reduced in numbers together with pathogenic streptococci like S. pyogenes. Unlike nisin A treatment, commensals species related to the genera Veillonella, Streptococcus, and Haemophilus enriched after salivaricin 10 treatment with the two tested concentrations (25 μg and 50 μg). Dental health is usually associated with a more significant proportion of streptococcal cells of beneficial characteristics such as S. gordonii, S. sanguinis and S. parasanguinis[24]. S. gordonii, S. parasanguinis were strongly inhibited by nisin exposure and were maintained at normal levels upon salivaricin 10 treatment (FIG. 6-c). These commensal streptococci were usually associated with good oral health, and their decreased colonization is associated with the progression of periodontal disease [25]. They also produce hydrogen peroxide (H₂O₂) to outcompete cariogenic pathogens [26]. Data presented here suggest that salivaricin 10, can modify the oral biofilm by reducing disease-associated bacteria while maintaining the number of essential commensals for a balanced oral microbiome. Such modulation may enable a better immune response and eventually reduce oral inflammation. Unlike salivaricin 10, nisin A disrupted the indigenous oral biofilm, including beneficial commensals using its broader spectrum of antimicrobial activity; this is one of the main problems hindering nisin A development to maintain a balanced oral microbiome.

Biofilms were formed ex-vivo using cell-free saliva as a medium and total salivary cell as inoculum (from healthy subjects). Twenty-four hours pre-formed multispecies biofilms grown anaerobically were treated with either nisin A (50 μg·ml⁻¹) or two concentrations of salivaricin 10 (25 and 50 μg·ml⁻¹) for a time exposure of 20 min. Upon treatment with salivaricin 10 (at both tested concentrations), like nisin A control, the confocal microscopy images confirmed significant and critical damage to the biofilm architecture. Salivaricin 10 penetrated saliva-derived multispecies biofilms and effectively killed the bacterial cells within (labeled with PI, lighter gray). No such effect was noticed with untreated (PBS) biofilms controls where the viable cells labeled mainly with Syto9, black. the ratio of live to dead cells decreased by a factor of 6 times compared to PBS treated conditions. Compared with PBS controls, salivaricin 10 at concentration of 50 μg·ml⁻¹ but not 25 μg·ml⁻¹ caused a reduction in the overall biofilm thickness. This dose-dependent reduction in the oral biofilm thickness was reported previously for nisin A [27]. Interestingly, the heterogeneity of bacterial distribution (roughness) measured by viable signal intensity of each pixel of the confocal microscopy images showed a 2-fold decrease upon treatment with salivaricin 10 compared to PBS, which indicates less variation in the distribution of bacteria within biofilms.

FIG. 6 summarizes the antibiofilm activity of salivaricin 10. FIG. 6a shows a schematic of the process followed. Collected saliva samples from healthy subjects were cultured in 24 imaging well plates to grow oral biofilms. The biofilms were treated with either PBS, nisin A or salivaricin 10 followed by washing steps as mentioned in the method section. The biofilms were then scraped off the wells and suspended in PBS to create an inoculum which was used to inoculate fresh BHI medium (1:100). The new sub-cultures were incubated overnight, and the grown cells were collected followed by DNA extraction and 16S rRNA gene sequencing using illumine MiSeq. In a duplicate experiment, Live/Dead molecular probes were used to determine killing effect of salivaricin 10 against pre-formed saliva-derived multispecies biofilm. Live cells stained with Syto 9 while dead (permeabilized) cells are labeled with (PI). FIG. 6b illustrates that salivaricin 10 modulate the multispecies biofilm towards commensal-rich microbiome. Salivaricin 10 improved total Firmicutes and Proteobacteria numbers while reduced Bacteroidetes significantly. 16S rRNA gene analysis at species level shows that salivaricin 10 maintained the number of commensal oral microbiota such as species related to the genera Veillonella, Haemophilus, Streptococcus while reduced Prevotella, Actinomyces and pathogenic streptococci. FIG. 6c shows representations of sum projections of Z-stack images taken by confocal microscopy. A dark black signal indicates viable live cells (Syto 9), a lighter gray signal indicates damaged/dead cells (propidium iodide). d, ratio of viable signal to dead signal was measured by Image J. e, Thickness of biofilm was measured after z-stack images from lower part of biofilm to highest part of biofilm (highest signal intensity). f, the heterogeneity of bacterial distribution (roughness) was measured by viable signal (bacteria labeled with Syto 9) intensity of each pixel using ImageJ.

### Salivaricin 10 Primes Innate Immune Cells to Augment Phagocytosis and Produce Anti-Inflammatory Response

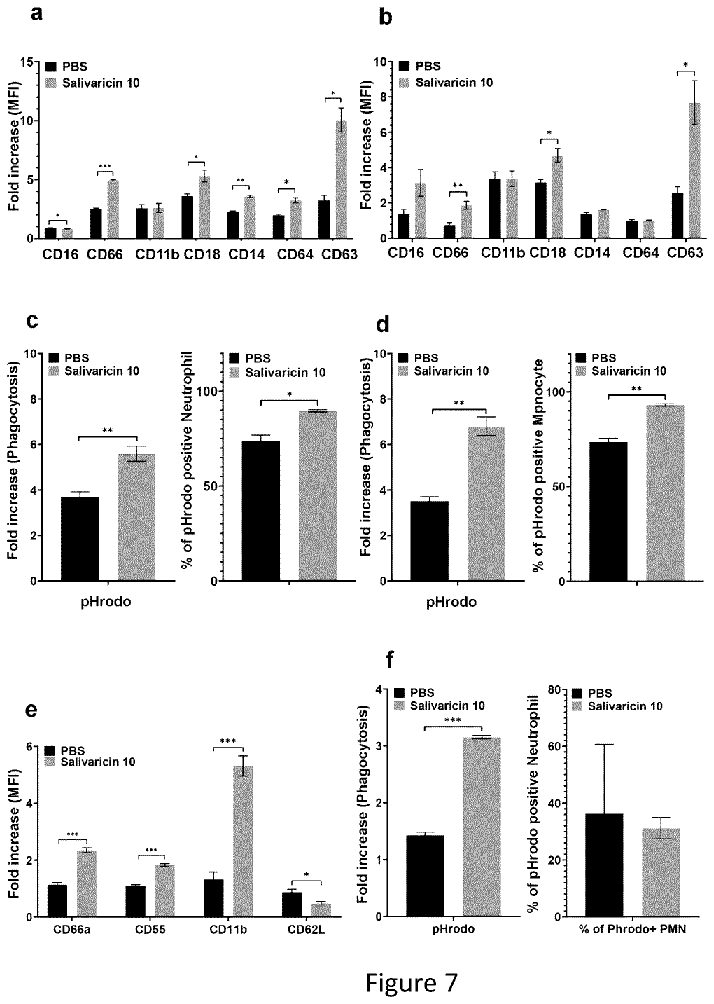

Neutrophils and monocytes display different activation states characterized by expression levels of cell surface CD marker in the oral cavity depending on the bacteria that they are interacting with. In health, neutrophils in the oral cavity are in a parainflammatory state, and with dysbiosis neutrophil phenotypes change towards a proinflammatory state [28, 29]. To determine if salivaricin 10 can prime neutrophils to augment pathogen elimination through phagocytosis, the surface expression of specific activation CD markers was assessed on human and murine neutrophils stimulated either with pHrodo (pH-sensitive, heat killed E. coli) alone or with salivaricin 10 and pHrodo. A phagocytosis assay was performed on whole blood neutrophils and monocytes (from human) and bone marrow suspension (from mice) and compared PBS and salivaricin 10 stimulation capacity both in presence of the same concentration of pHrodo. Salivaricin 10, concentration used 50 μg·ml⁻¹, upregulated surface expression of CD66, CD18, CD14, CD64 and CD63 in neutrophils (FIG. 7-a), CD66, CD18 and CD63 in monocytes (FIG. 7-b). In addition, phagocytosis by neutrophils and monocytes increased significantly with salivaricin 10 (FIGS. 7-c and d). Mice bone marrow neutrophils were also primed by salivaricin 10 in the phagocytosis assay and CD markers CD11b, CD55 and CD66a were upregulated together with induction in the fold increase in phagocytosis per neutrophil (pHrodo fluorescence) (FIG. 7-e) but not the total number of phagocytic cells (FIG. 7-f).

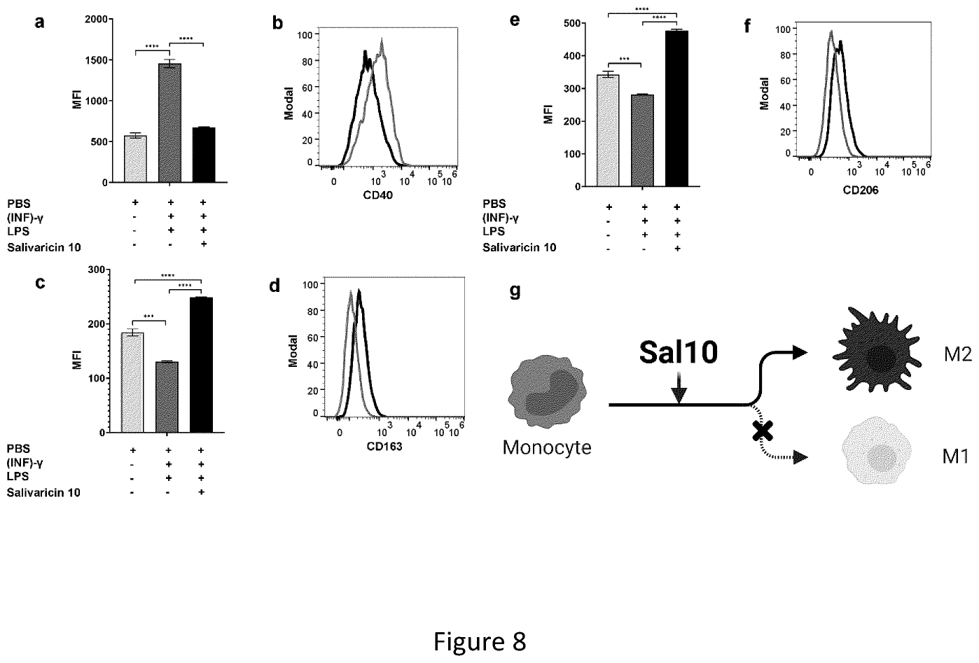

To evaluate if salivaricin 10 can directly interact with macrophages, thp1 monocytes were polarized in vitro to a pro-inflammatory, M1 phenotype. The addition of salivaricin 10 in cell culture media was found to reverse the polarization and promoted a pro-resolution, M2 phenotype (high CD206 and CD163). FIG. 8 summarizes the results, which show that Salivaricin 10 polarizes monocytes towards M2 pro-resolution phenotype. Thp1 monocytes were differentiated with LPS+INF-γ, in the presence or absence of Salivaricin 10 (Sal10). The peptides were found to block M1 polarization, while promoting expression of pro-resolution (M2) macrophage markers (g). In FIG. 8, the gray line shows cells stimulated with IFN and LPS (M1); the black line shows cells stimulated with IFN, LPS and then salivaricin 10 to alter polarization. FIGS. 8a, c, and e show analysis of CD40, CD163 and CD206 respectively. FIGS. 8b, d, f: representative flow spectrum of each CD marker.

We investigated whether salivaricin 10 injection into the peritoneum alters the phagocytic activity of macrophages and neutrophils in the peritoneal lavage. pHrodo E. coli BioParticles conjugates were injected concomitantly with either PBS or salivaricin 10, and phagocytosis was assessed after 3 hours. Salivaricin 10 administration increased the phagocytic activity per neutrophil and the number of phagocytic positive neutrophils in mice compared to PBS-treated control animals (p<0.05).

### Salivaricin 10 Modulates the Phagocytic Activity of Peritoneal Neutrophils

We investigated whether salivaricin 10 injection into the peritoneum alters the phagocytic activity of macrophages and neutrophils in the peritoneal lavage. pHrodo E. coli BioParticles conjugates were injected concomitantly with either PBS or salivaricin 10, and phagocytosis was assessed after 3 hours. Salivaricin 10 administration increased the phagocytic activity per neutrophil and the number of phagocytic positive neutrophils in mice compared to PBS-treated control animals (p<0.05).

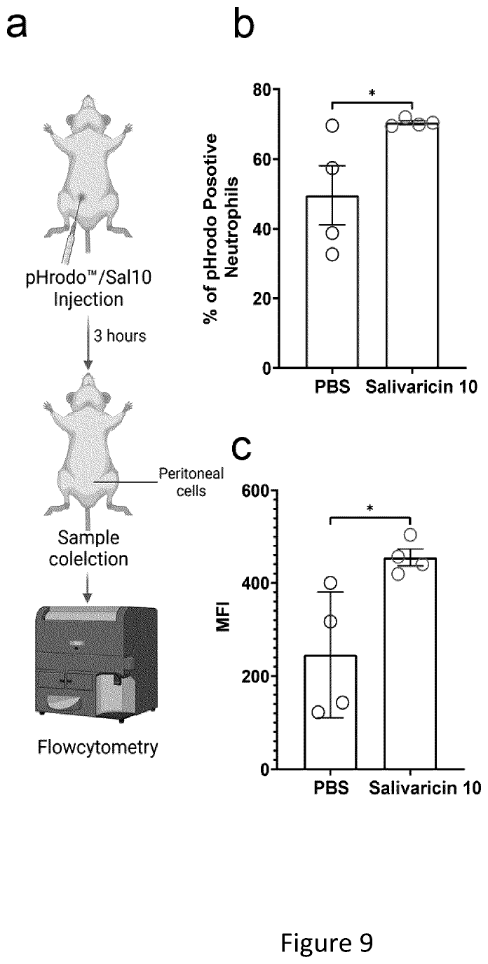

FIG. 9 summarizes the results: Salivaricin 10 improve phagocytosis in vivo. FIG. 9a, pHrodo E. coli BioParticles conjugates were injected concomitantly with either PBS or salivaricin 10 into the peritoneal cavity. FIG. 9b, percentage of phagocytic neutrophils. FIG. 9c, total signal of phagocytosis in neutrophils.

### Salivaricin 10 is a Neutrophil Activator of Reactive Oxygen Species (ROS) and Chemotaxis

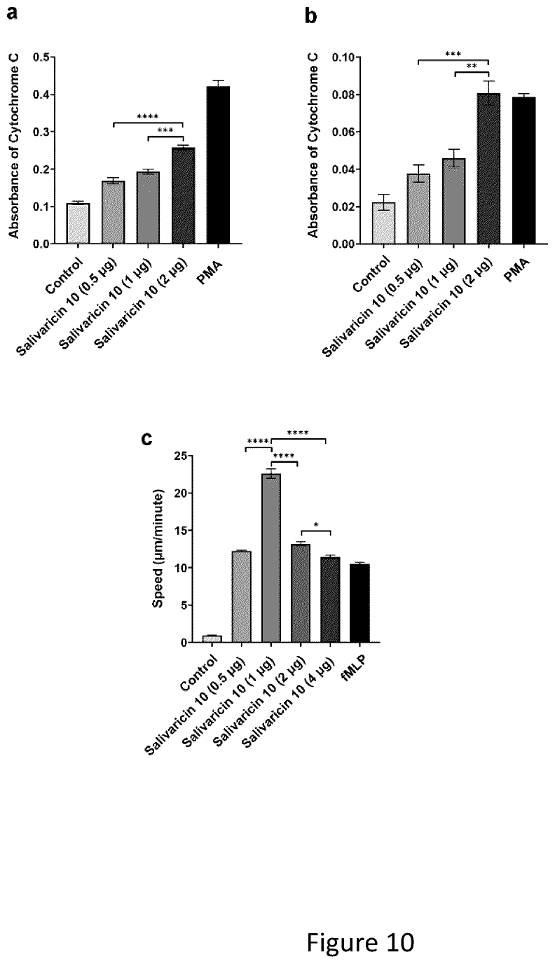

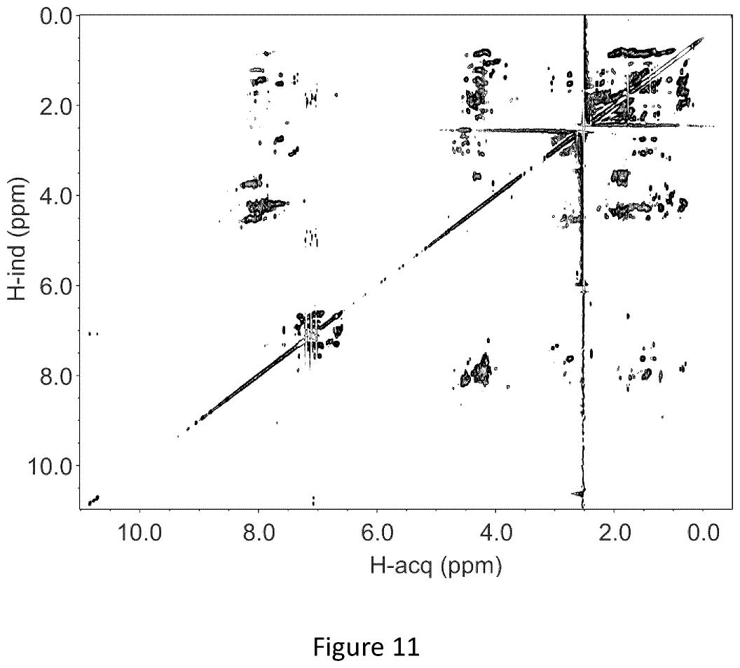

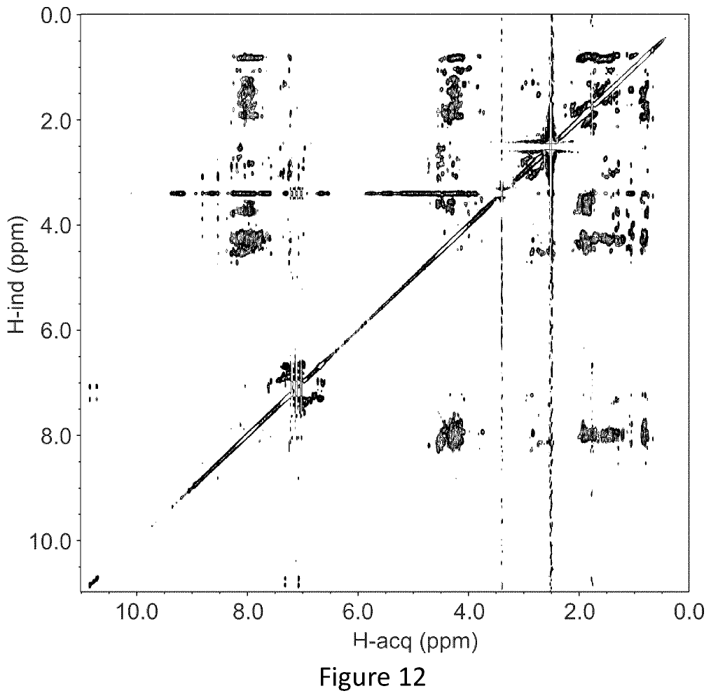

Salivaricin 10 was found to activate ROS production in a concentration dependent manner in both human and murine neutrophils (FIG. 10-a,b). Neutrophils produce ROS at the site of infection following activation of surface receptors, the produced ROS then cross the bacterial pathogens membranes and damage their nucleic acids, proteins, and cell membranes[30]. Formylated peptides are an important neutrophil chemoattractant associated with bacterial pathogens. These peptides can activate innate immune cells by binding to formyl peptide receptors (FPR) to stimulate chemotaxis and phagocytosis towards pathogens. Salivaricin 10 induced immunomodulatory activities in neutrophils; further, phosphorylated lanthipeptides of salivaricin 10 were found to be neutrophils chemoattractants. Chemotaxis assay with isolated human neutrophils were performed, and neutrophil migration speed due to addition of increasing concentration of salivaricin 10 was measured. The control peptide used in this assay, N-formyl-methionyl-leucyl-phenylalanine (fMLP), induced neutrophils chemotaxis to ˜11 μm/minute. Salivaricin 10 showed similar effect with all concentrations tested (2.5, 5, 10 and 20 μg). Noteworthy, salivaricin 10 at a concentration of 5 μg induced a 2-fold increase in speed compared to ƒMLP (FIG. 10-c).

### Acyclic Phosphorylated N-Terminal Peptide of Salivaricin 10 Contains Important Immunomodulation Activities

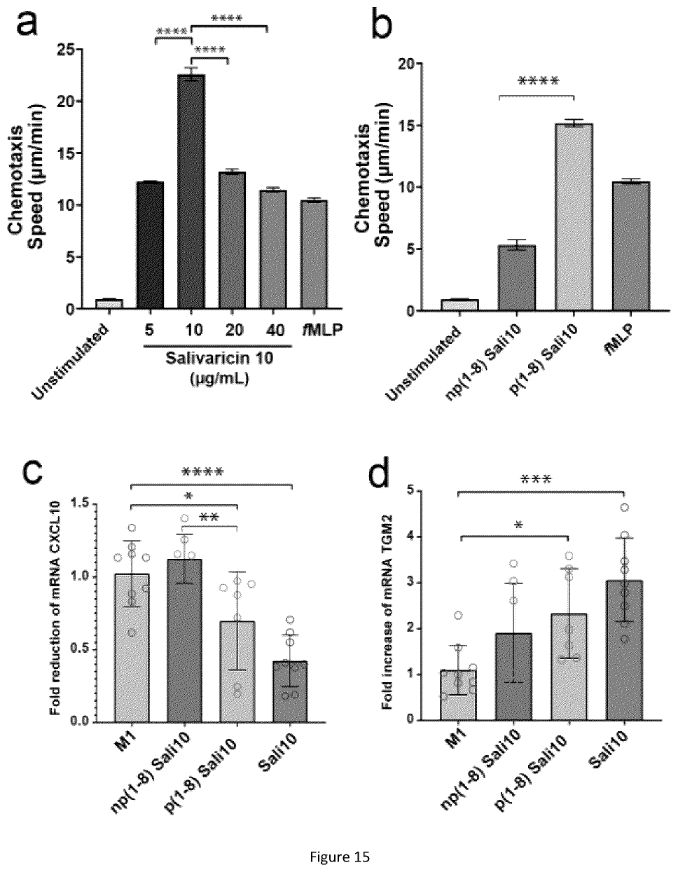

To test whether the phosphorylated N-terminal tail of SrnA1 and SrnA2 is important for the observed immunomodulatory activity, we synthesized the eight N-terminal unbridged residues NH₂-GWFTAVQL-COOH, with the phosphorylated (pThr4) (SEQ ID NO: 16). This short peptide analogue, named p (1-8) Sali10 (SEQ ID NO: 16), induced chemotaxis by neutrophils with very similar activity to the full-length salivaricin 10 (FIG. 15b), supporting the importance of the N-terminal tail in sensitizing neutrophils. The chemotaxis of neutrophils treated with a non-phosphorylated analogue of the N-terminal tail [np(1-8) Sali10] was significantly reduced, suggesting that the phosphate group at position 4 (pThr4) is important for the observed immunomodulatory activity. We also found that both full-length salivaricin 10 and p(1-8) Sali10 (SEQ ID NO: 16), but not the non-phosphorylated np (1-8) Sali10, downregulated the gene expression of the C—X—C motif ligand-10 gene (CXCL10), which is one of the main biomarkers of M1 pro-inflammatory macrophages (Figure. 15c). Both full-length salivaricin 10 and p(1-8) Sali10 (SEQ ID NO: 16) peptides, but not np (1-8) Sali10, upregulated the gene expression of TGM2,M2 anti-inflammatory macrophage biomarker, significantly compared to M1 (control cells) (Figure. 15d). However, there was no significant difference in the gene expression of TGM2 between p (1-8) Sali10 (SEQ ID NO: 16) and np (1-8) Sali10 peptide treatments. Neither p (1-8) Sali10 (SEQ ID NO: 16) nor np (1-8) Sali10 showed effects on the neutrophils' CD marker expression, unlike full-length salivaricin 10, suggesting that the full length or a component of the full peptides is very important for stimulating these other immunomodulatory activities. The data provides strong evidence that the N-terminal 8 amino acids of salivaricin 10, particularly the unique phosphorylated threonine residue (pThr4), are important for the immunomodulatory properties of this lanthipeptide.

### Discussion

RiPPs are a growing class of natural products with drug-like characteristics. Salivaricin 10 is a phosphorylated tripeptide system composed of lanthipeptides with antibacterial, antibiofilm and immunomodulatory activities produced by the SALI-10 S. salivarius strain. Phosphorylated lanthipeptides from wild-type bacterial strains have not been reported previously, although phosphorylation is common in signal-transducing proteins in prokaryotes and eukaryotes. The salivaricin 10 synthetase, SrnM-10, catalyzes three dehydrations and three cyclization reactions in the pre-salivaricin peptides. SrnM-10 incorporates one phosphate into the salivaricin 10 peptides via phosphorylation reaction during Ser/Thr dehydration.

We observed higher molecular weights (˜+80 Da) of salivaricin 10 peptides than predicted, suggesting that all the three lanthipeptides are phosphorylated. This was confirmed by NMR and high-resolution MS/MS data as one phosphate was observed during structural analysis. This can be explained by the unique sequence of SrnM-10 (i.e. the SrnM gene of SALI-10), which is responsible for phosphorylation and phosphate elimination. The in-silico analysis of translation of the srnM-10 gene revealed four mutations in the resultant translated protein (SrnM-10 protein, SEQ ID NO:9) compared to the srnM gene reported for other S. salivarius strains. These mutations include Ser5 (SrnM-10)/P5 (SrnM-JH), Met590 (SrnM-10)/Ile590 (SrnM-JH), Thr712 (SrnM-10)/Ala712 (SrnM-JH) and Phe739 (SrnM-10)/Ala739 (SrnM-JH). Four of these mutations are within the C-terminal domain of the SernM enzyme anticipated to be responsible for cyclization. Interestingly, the phosphorylation retained excellent antimicrobial activity for salivaricin 10.

Noteworthy, the amino acid composition of the three lanthipeptides did affect the overall antimicrobial activity. The major peptide (SrnA2) produced a more extensive zone of inhibition against a lantibiotic-sensitive target, M. luteus. The activity was reduced with SrnA1, which only differed in one amino acid at position 22 compared with SrnA2. Reduced antimicrobial activity was detected for the third peptide, SrnA4, (in isolation) compared with SrnA1 and SrnA2.

Homologue genes encoding the three peptides of salivaricin 10 were previously found in an S. pneumoniae strain P174 with limited antimicrobial activity [DOI: https://doi.org/10.1128/mBio.01656-15]. Functional analysis of the genes harbored on the S. pneumoniae chromosome was carried out by gene knockout and complementation study. It was shown that deletion of any of the three genes would result in abolished antimicrobial activities. On the contrary, the HPLC-purified peptides of salivaricin 10 in the current study showed distinct antimicrobial functions. This can indicate that the peptides produced by S. pneumoniae strain P174 were not successfully post-translationally modified to induce distinct bioactivities.

While S. salivarius SALI-10 is the first bacterial strain to produce the three peptides successfully, S. salivarius JH strain was reported previously to produce one peptide, named salivaricin E, sharing the same amino acid sequence of peptide 1 of salivaricin 10 of the current study. Salivaricin E was reported to possess antimicrobial activity against Streptococcus mutans, and to have a molecular weight of 3565.9 Da (with four dehydrations) [DOI: 10.1099/mic.0.000237]. This mass is 112 Da higher than the predicted 3454 Da of successfully translated peptide with four dehydrated residues. Noteworthy, pairwise sequence alignment of the SrnM-10 lanthionine synthetases of strains SALI-10 and JH showed four mutations.

Since the commensal S. salivarius produces all three peptides, we called it a tri-peptide system, and we tested this composition in all downstream applications. Salivaricin 10 was able to penetrate saliva-derived biofilms and kill bacterial cells within.

The dental biofilm consists of bacteria embedded in a proteinaceous and polysaccharide-rich matrix, and these biological barriers make it difficult for therapeutic agents to penetrate and reach the targeted bacterial cells. The biofilm's bacterial constituents high resistance to host immune responses and antimicrobials, complicating treatments and sometimes leading to life-threatening conditions [31]. Salivaricin 10 has an ability to penetrate oral biofilms and kill bacteria within by pore formation.

The protective potential of salivaricin 10 may include a symbiotic relationship with innate immunity that promotes the host defense by enhancing phagocytic activity of neutrophils and macrophages and fostering a more rapid clearance of the bacterial load resulting in faster inflammation resolution. This may be further mediated by the peptide as we have shown that the peptide encourages M2 macrophage differentiation. Bacterial pathogens have evolved biological tools to avoid detection by innate immunity first-line responders. For instance, the oral and systemic pathogen P. gingivalis suppresses macrophage immune functions by synergizing with C5a (a protein fragment released from cleavage of complement component C5 by protease C5-convertase) to increase cyclic adenosine monophosphate (cAMP) concentrations [32]. Moreover, the significant human pathogen S. pyogenes can persist into the host upper-respiratory-tract by circumventing the host defence mechanisms to exploit the inflammatory response. Such mechanisms enable S. pyogenes to survive in phagocytic neutrophils and take advantage of the leukocyte trafficking to be transported and spread from primary sites of infection or even expressing DNase to degrade neutrophil extracellular traps (NETs) [34]. Other oral pathogens like S. pneumoniae can evade neutrophils phagocytosis by activating NF-κB via its cell wall anchoring protein PfbA [35]. Salivaricin 10 not only inhibits S. pyogenes and S. pneumoniae potently by direct killing but also acts as a potent neutrophil chemoattractant to improve recruitment of phagocytic cells and cooperate with the innate immune response for pathogen clearance. There is increasing interest in developing antibiotics as neutrophil chemoattractant in order to increase antimicrobial efficacy.

In summary, the results of this study show for the first time that phosphorylated lanthipeptides from the commensal oral microbiome can confer multi-level protection to the host against bacterial infections. Salivaricin 10 peptides can act as antibiotics and antibiofilm agents and induce immunomodulatory effects by communicating with innate immune cells that regulate anti-inflammatory and pro-resolution responses. Phosphorylation of salivaricin 10 is required to induce effective neutrophil chemotaxis and for its regulation of inflammation resolution through M2 macrophage differentiation. The phosphorylated mature form of the salivaricin 10 peptides sets them apart from all other lanthipeptides studied to date. Our study sets the stage for future development of salivaricin 10 as an oral antimicrobial therapeutic that has multiple distinct bioactivities, i.e., penetrating biofilms, killing pathogens while sparing commensal streptococci, and stimulating inflammation and its resolution.

### Methods

### 1. Isolation and Screening of Bacteriocin-Producing Strains.

Clinical isolates of S. salivarius were obtained either from saliva or from the dorsum surface of the tongue of healthy subjects using sterile cotton swabs. The samples were serially diluted in phosphate buffer saline (PBS) at pH 7 before plating on Mitis Salivarius Agar (Difco) followed by incubation at 37° C. with 5% CO₂ in air to grow S. salivarius colonies selectively. The inclusion criteria of the subjects included in this study are as follows; a) healthy, b) 18-30 years old, c) males and females. Exclusion criteria included a) subjects with dental problems e.g., tooth decay and periodontal diseases, b) subjects with upper respiratory tract infections, c) subjects with anemia and d) subjects with a sore throat. Typical morphological S. salivarius colonies grown on MSA were picked using sterile inoculation loops and used to inoculate 5 mL of Tryptic Soy Broth (Difco) supplemented with 0.8% Yeast Extract (Difco). The cultures were allowed to grow at the same incubation conditions mentioned above before glycerol stocks (final concentration 15% glycerol) were made and stored at −80° C. Screening for bacteriocin production was performed using deferred antagonism assay as mentioned previously using Group A Streptococcus, Group B Streptococcus, Streptococcus mutans, Streptococcus oralis and Micrococcus leutus as the targeted indicator strains. Strains exhibiting inhibitory activity against at least one indicator were considered as bacteriocin producers. Colony PCR was performed on all isolated strains using gtfK primers for S. salivarius identification and sets of primers to screen for most common salivaricins A, B, 9, G32 and streptin as mentioned previously [38]. Strains which are negative to the screened salivaricins but still show potent inhibitory activity were selected for further analysis to simplify the purification process of less characterized bacteriocins e.g., salivaricin E. Strains with potent antimicrobial activity against the indicator strains were selected for further peptide extraction and inhibitory spectrum analysis using a panel of antibiotic resistant strains (Table 9).

2. Complete Genome Sequencing of S. salivarius SALI-10.

S. salivarius SALI-10 was grown in 35 mL BHI (difco) using the same conditions mentioned above. The cells were collected by centrifugation at 4,000×g and resuspended in 5 mL TE buffer. The suspension was combined with lysozyme at a final concentration of 15 mg and incubated at 37° C. for 1 h. Six hundred microliters of EDTA and 100 μL proteinase K (QIAGEN™, USA) were then added to the suspension and incubated again at 37° C. for 1 h. Seven hundred microliters of 12% SDS were then added to the suspension and incubated at 50° C. for 2 h on an orbital shaker (150 rpm). Cold potassium acetate was added, and the sample was centrifuged at 14,000×g for 15 min. The supernatant was collected and combined with RNAse A (final concentration 50 μg/mL) and incubated at 37° C. for 1 h. One volume of cold isopropanol was added to the sample to precipitate the intact high molecular genomic DNA, which was fished out using a glass rod. The DNA was washed with 70% EtOH and dissolved in PCR-grade EDTA-free water. Purified genomic DNA was quantified using the TapeStation™ DNA system (Agilent, Canada). The DNA library was prepared according to Pacific Biosciences 20-kb template preparation protocol using the BluePippin™ size-selection system. Qualified genomic DNA was fragmented using the Covaris g-TUBE device and then end-repaired to prepare SMRTbell™ DNA template libraries (with a fragment size of 15 kb to 50 kb). Complete genome sequencing was performed using a PacBio Sequel II, Single Molecule, Real-Time (SMRT) 8M sequencing (The Centre for Applied Genomics, The Hospital for Sick Children, Canada) using 15 hours movie time to ensure complete genome sequencing with no gaps. Raw BAM files were used for the genome assembly using hierarchical genome-assembly process (HGAP) for high-quality de novo microbial genome assemblies [39]. Genome annotation was carried out by the SEED and the Rapid Annotation of microbial genomes using Subsystems Technology (RAST) [40].

### 3. Identification of Biosynthetic Gene Cluster (BGS).

The complete genome of S. salivarius SALI-10 obtained by PacBio sequencing was screened by antiSMASH secondary metabolite genome mining pipeline, bacterial version [41]. The complete genome of SALI-10 was as follows: (SEQ ID NO:14: genome; SEQ ID NO: 15: megaplasmid):

### 4. Strain Fermentation and Purification of the Salivaricin 10 Peptides.

Pure and homogenized SALI-10 colonies were selected to inoculate 10 ml of TSB supplemented with 2% yeast extract. The fermentation was carried out overnight at 37° C. with 5% CO₂ for 18 h. The produced culture was used to inoculate 20 Petri dishes (100 mm), each containing 20 mL of TSA supplemented with 2% yeast extract and 0.2% CaCO₃ and incubated as mentioned above (step 1). The agar cultures were then cut into small pieces and put into 250 mL centrifugation bottles. The bottles were incubated at −80° C. for 18 h and thawed immediately in a 70° C. water bath to produce liquid material. The liquid material was then collected by centrifugation, and the resultant supernatant was extracted with one volume of chloroform. The white precipitate from chloroform extraction was dried and resuspended in 2 ml of 35% acetonitrile in water (v/v). Centrifugation was performed to remove insoluble material to produce a clear peptide extract (we call it material A). The process was repeated with induction procedure by adding 50 μl of material A to 50 ml of TSB supplemented with yeast extract and inoculating this media with SALI-10 strain by repeating step 1 and generating 300 induced agar cultures instead of 20. The induced and chloroform extracted peptide was dried as in step 1 and resuspended in 20 ml of 35% ACN in water (v/v) supplemented with 0.1% TFA. The peptides preparation was processed via solid-phase extraction using C18 Sep-Pak® column (Waters, USA) prewashed with methanol and water. After loading the peptides sample, the column was washed with 10 mL of increasing methanol concentrations (10%, 30%, 50%, 70%). Active peptides were eluted with 95% methanol supplemented with 0.1% TFA. The peptides were finally purified using HPLC and C18-Reversed Phase chromatography as mentioned previously [42]. Eluted fractions (1 ml per minute) were tested for antimicrobial activity against the sensitive strain M. luteus ATCC10240 using the spot-on-lawn method.

### 5. Identification and Characterization of Salivaricin 10 Peptides.

Initial mass determination of the peptide products prepared either from cell extracts or semi-purified peptide preparation was performed using electrospray ionization quadrupole time of flight mass spectrometry (ESI-QTOF-MS) with a Xevo G2-XS mass spectrometer (Waters, Mississauga, ON, Canada). The raw data were processed using a MassLynx software system (Waters, Mississauga, ON, Canada).

The liquid chromatography-tandem mass spectrometry (LC-MS/MS) system used on the purified products was comprised of a Shimadzu Nexera X2 MP Ultrahigh-Performance Liquid Chromatography system (Kyoto, Japan) coupled to a Thermo Fisher Scientific high-resolution Orbitrap Fusion™ Tribrid™ mass spectrometer (MS). Operational control of the LC-MS/MS system was performed with the following software packages from Thermo Fisher Scientific: Xcalibur for data acquisition; FreeStyle for chromatographic peak and MS/MS spectral analysis; and, TraceFinder for peak integration. LC separations were performed using mobile phase (MP) solutions consisting of 0.1% FA in water (MPA) and 0.1% FA in ACN (MPB), and a needlewash solution consisting of ACN:water (1:1, v: v). The chromatographic method included ambient column and autosampler tray temperatures, a mobile phase flowrate of 0.200 mL/min, and a gradient elution program specified as follows: 0-0.5 min, 30% MPB; 0.5-5.5 min, 30-60% MPB; 5.5-6.5 min, 60-100% MPB; 6.5-8.0 min, 100% MPB; 8.0-8.5 min, 100-30% MPB; 8.5-12.5 min, 30% MPB. The overall cycle-time for a single injection was approximately 13.0 min.

NMR analysis was performed as described previously [43]. Approximately 1.5 mg of combined HPLC fractions of SrnA1 (phe-variant) and SrnA2 (tyr-variant) were dissolved in 600 μl of deuterated dimethyl sulfoxide (DMSO-d6) and the NMR data were collected on a Bruker Advance III-HD spectrometer operating at a proton frequency of 850 MHz. The 1H resonances were assigned according to standard methods [44] using correlation spectroscopy (COSY), TOCSY [45], NOESY experiments. NMR experiments were collected at 25° C. The TOCSY experiment was acquired with a 60-ms mixing time using the Bruker DIPSI-2 spinlock sequence. The NOESY experiment was acquired with 400- and 500-ms mixing times. Phase-sensitive indirect detection for NOESY, TOCSY, and COSY experiments was achieved using the standard Bruker pulse sequences. Peaks were assigned using NMRView software [47].

### 6. Colony Mass Spectrometry for Salivaricin 10 Distribution.

Four to five colonies, grown on TSYECa agar plates, of each S. salivarius isolate, were collected using a sterile inoculation loop and resuspended in 250 μL of 70% isopropanol supplemented with 0.1% TFA. The cell suspensions were vortexed vigorously for 15 minutes then incubated on ice for 2 hours. The cell suspensions were then centrifuged at 18,000×g for 30 minutes, and the supernatant was collected and subjected to LC-MS analysis. The Salivaricin 10 three peaks were detected in strain SALI-10 and identified as mentioned above with the exact molecular weights. Salivaricin 10-negative variants were identified with the same peptide spectrum with strain SALI-10 but lacked the three peptide peaks corresponding to Salivaricin 10.

### 7. Minimum Inhibitory Concentration (MIC).

The purified fraction containing the three peptides composing salivaricin 10 was dried, weighed on an analytical balance and suspended in PBS. MIC was performed as mentioned previously [48]. The MIC is the lowest concentration of compound that inhibits the visible growth of the bacteria after 24 h of incubation at 37° C. TSA media was used to propagate the indicator strains. 5% Sheep blood was added to some bacterial cultures, e.g., S. pneumoniae, to promote growth.

### 8. Time Killing Assay of Salivaricin 10.

Tryptic soy broth was inoculated 1:100 with overnight cultures of either S. pyogenes W51503011 or E. faecalis ATCC29212 and was incubated at 37° C. without shaking until cells were grown to 1×10⁶ c.f.u. per ml. Then, 10×MIC salivaricin 10 was added. At the time points of 0 h, 2 h, 4 h, 6 h and 24 h, samples were taken and serially diluted in PBS. Twenty microliters of each dilution were spotted on tryptic soy agar plates and allowed to dry, and colony counts were determined after overnight incubation at 37° C. using the following equation c.f.u. per ml=the Average number of colonies for a dilution×50× dilution factor.

### 9. Kinetics Pore Formation Assay.

Membrane permeability assay was carried out using the DNA dye Sytox Green (Invitrogen). Cultures of M. luteus s ATCC10240 were grown overnight at 37° C. in TSB medium on an orbital shaker and then diluted with fresh TSB to 1×10⁶ c.f.u. per ml. Cells were combined with Sytox Green (final concentration 5 μM), HEPES (1 mM) and glucose (1 mM) and incubated in the dark for 10 minutes at room temperature. Labelled cells were analyzed using Cytation 5 plate reader (BIOTEK) for 10 min to establish a stable baseline of the fluorescent intensity (FI). Nisin A (10×MIC), salivaricin 10 (5 and 10×MIC) or PBS were added to the cells and the FI was monitored for 30 min with time interval of 1 min for fluorescence measurement at 488/523 nm excitation/emission. A rapid increase in FI upon antibiotic addition is a strong indication of membrane permeabilization as Sytox Green is impermeable to intact membranes.

### 10. Membrane Potential Dissipation

Membrane potential dissipation assay was carried out using the BacLight™ Bacterial Membrane Potential Kit (Invitrogen). Cultures of M. luteus s ATCC10240 were grown overnight at 37° C. in TSB medium on an orbital shaker and then diluted with fresh TSB to 1×10⁶ c.f.u. per ml. Cells were combined with DiOC2 (3) according to the manufacturer instructions. Labelled cells were analyzed using Cytation 5 plate reader (BIOTEK) for 2 min to establish a stable baseline of the fluorescent intensity (FI). Nisin A (10×MIC), salivaricin 10 (5 and 10×MIC) or PBS were added to the cells and the FI was monitored for 30 min with time interval of 1 min for fluorescence measurement at 482/497 nm excitation/emission. A rapid increase in FI upon antibiotic addition is a strong indication of the membrane depolarization due to pore formation.

### 11. Intracellular Accumulation of the Peptidoglycan Cell Wall Precursor (UDP-MurNAc-Pentapeptide)

Accumulation of the UDP-MurNAC-pentapeptide inside targeted bacterial cells was analyzed as described previously with some modifications. M. luteus ATCC10240 cells were grown in BHI on an orbital shaker at 150 rpm, 37° C. for overnight. The culture was diluted 1/20 (v/v) using a fresh medium and further incubated under the same conditions mentioned above until OD₆₀₀ reached 0.5. Chloramphenicol was added at a final concentration of 130 μg/mL and further incubated for 15 minutes. Then the culture was divided into four equal samples (5 mL each) into four 15 mL sterile tubes. Vancomycin was added to the first tube at 10×MIC. The second tube was supplemented with salivaricin 10 (5×MIC), and the third tube was supplemented with salivaricin 10 (10×MIC). The fourth sample was left untreated and served as a control. The four samples were incubated for 40 minutes before centrifugation at 3,000 rpm for 30 minutes. The supernatant was discarded, and the cell pellets were extracted with boiling water for 20 minutes. Cell debris was removed by centrifugation at 13,000 rpm for 15 minutes, and SpeedVacuum concentrated the supernatant to 0.5 mL. Intracellular accumulation of UDP-MurNAc-pentapeptide was analyzed by UPLC-MS using acetonitrile gradient in water on the C18 column at a flow rate of 1 ml·min⁻¹. Diode chromatograms and Tof-MS profiles were compared between the samples to analyze the accumulation of the cell wall precursor.

### 12. Antimicrobial Activity Against Preformed Biofilms (Saliva-Derived De Novo Model)

Saliva-derived biofilms were established in 24 well glass bottom sensoplates (Greiner Bio-One, Monroe, NC, USA) as mentioned previously [27]. The formed biofilms were then challenged with either salivaricin 10 or nisin A for 30 min, and PBS was used as a negative control. The peptides were removed by pipette, and the biofilms were washed twice with PBS. The biofilm cells were then labelled with Filmtracer LIVE/DEAD Biofilm Viability Kit (Invitrogen, USA), following the manufacturer's instructions. The stains were removed, and the biofilms were washed twice with PBS before fixation using 1.6% paraformaldehyde (PFA). The PFA was removed then Fluoromount-G Mounting Medium (Invitrogen, USA) was added to thin coverslips before flipping over each biofilm sample. The samples were left to mount overnight in the dark at room temperature. Microscopy and imaging were carried out as follows: all biofilms were acquired by a Zeiss LSM 800 with the Plan Apochromat objectives featuring Zeiss 20× (Plan Apochromat; 20×/0.8). 3D biofilm images were constructed by ZEN software (Zeiss). The mean Intensity of pixels for viable signals (Syto-9; live) and (PI; dead) were measured within biofilm areas of each image with the slices containing the highest respective signal intensity using custom Fiji (ImageJ). The thickness of biofilms was defined using a green channel (the distance between the lowest signal intensity from the bottom of biofilms to the highest signal intensity from the top of biofilms). Roughness calculation plugin from custom Fiji (ImageJ) was used to measure the heterogeneity of bacterial distribution based on green channel (Syto-9; live). The inputs were z-stacks in which the pixel values represent the distance to the surface, and the average of roughness was reported as arithmetical mean deviation (RA). All quantifications and statistical calculations are based on 10-15 images and 5-10 stacks for each repeat (three repeats). Image analyses were performed on a laptop computer equipped with an Intel Core i7 CPU, 64-bit operating system.

A duplicate of the above biofilms was processed differently. After peptide treatment and PBS washing as mentioned above, biofilms were suspended in 100 μL of PBS and biofilm suspension of each well was then used to inoculate 10 ml of BHI broth before incubation at 37° C. for overnight anaerobically. The grown cultures were then centrifuged at 4000×g for 15 min and the supernatant was discarded. DNA extraction was performed on the cell pellets using DNeasy PowerSoil Pro Kit (Qiagen) according to the manufacturer's instruction. The DNA was subjected to 16S rRNA gene sequencing.

13. 16S rRNA Gene Sequencing

To elucidate the antimicrobial action of salivaricin 10 on preformed salivary biofilms, we extracted the DNA of treated biofilms and subjected it to PCR amplification by targeting the V4 hypervariable region of the 16S rRNA gene, which was amplified using 515F and 806R primers to allow for multiplexing [49]. Amplification reactions were performed using 12.5 μL of KAPA2G Robust HotStart ReadyMix (KAPA Biosystems), 1.5 μl of 10 μM forward and reverse primers, 7.5 μL of sterile water and 2 μl of DNA. PCR conditions were 95° C. for 3 min, 22× cycles of 95° C. for 15 seconds, 50° C. for 15 seconds and 72° C. for 15 seconds, followed by a 5 min 72° C. extension. All amplification reactions were done in triplicate to reduce amplification bias, pooled, and checked on a 1% agarose TBE gel. Pooled triplicates were quantified using PicoGreen and combined by even concentrations. The library was then purified using Ampure XP beads and loaded onto the Illumina MiSeq for sequencing, according to manufacturer instructions (Illumina, San Diego, CA). Sequencing was performed using the V2 (150 bp×2) chemistry. A single-species (Pseudomonas aeruginosa DNA), a mock community (Zymo Microbial Community DNA Standard: https://www.zymoresearch.de/zymobiomics-community-standard) and a template-free negative control were included in the sequencing run.

### 14. Analysis of the Bacterial Composition of the Biofilm Samples.

The UNOISE pipeline, available through USEARCH v11.0.667 and vsearch v2.10.4, was used for sequence analysis. The last base was removed from all sequences using cutadapt v.1.18. Sequences were assembled and quality trimmed using -fastq_mergepairs with a -fastq_trunctail set at 2, a -fastq_minqual set at 3, a -fastq_maxdiffs set at 5, a -fastq_pctid set at 90, and minimum and maximum assemble lengths set at 243 and 263 (+/−10 from the mean) base pairs. Assembled sequences were quality filtered using -fastq_filter with a -fastq_maxee set at 1.0. The trimmed data was then processed following the UNOISE pipeline. Sequences were first de-replicated and sorted to remove singletons, then denoised and chimeras were removed using the unoise3 command. Assembled sequences were mapped back to the chimera-free denoised sequences at 99% identity OTUs. Taxonomy assignment was executed using SINTAX, available through USEARCH, and the UNOISE compatible Ribosomal Database Project (RDP) database version 16, with a minimum confidence cutoff of 0.8 [50]. OTU sequences were aligned using align_seqs.py v.1.9.1 through QIIME1 [51]. Sequences that did not align were removed from the dataset and a phylogenetic tree of the filtered aligned sequence data was made using FastTree [52]. The 16S copy number and V4 primer differences were estimated with the SINAPS algorithm and the UNBIAS reference databases, accessed through USEARCH.

### 15. Flow Cytometry Analysis of Peptide-Stimulated Neutrophils

To detect the changes in neutrophil and monocyte CD marker expression and phagocytosis an in vitro assay was performed using either whole human blood or murine bone marrow. For the human blood assay, 100 μl of blood samples were treated with Salivaricin 10 and incubated at 37° C. for 30 minutes, untreated samples served as control. Samples were then infected with 25 μl of pHrodo (Green Escherichia coli BioParticles conjucate, Invitrogen) followed by incubation for another 30 min at 37° C. Samples were fixed with 1.6% paraformaldehyde (PFA) for 15 min at 4° C. Cells were lysed by 1×BD Pharm Lysed solution and resuspended in fluorescent-activated cell sorting buffer (Hanks' balanced salt solution, 1% bovine serum albumin, 2 mM EDTA). Rat serum (60 to 80 μg; Sigma) and mouse IgG (2 μg; Sigma) were added to the samples for 20 minutes on ice to block non-specific binding of immunoglobulins/antibodies to the Fc receptors. Cells labeling was carried out with the following antibodies: CD16-Alexa Fluor 700 (BioLegend), CD63-peridinin chlorophyll protein (PerCP)-Cy5.5 (BioLegend), CD66-allophycocyanin (APC) (eBioscience), CD14 PE-Cy7 (BioLegend), CD18-brilliant violet 421 (BV421) (BD), CD11b-APC-Cy7 (BioLegend) and CD64-phycoerythrin (PE) (BD). The same procedure was used for mouse bone marrow assay and the samples were labeled with the following antibodies, F4/80-brilliant violet 421 (BV421) (BioLegend), Ly-6G-peridinin chlorophyll protein (PerCP)-Cy5.5 (BD), CD62L-APC-Cy7 (BioLegend), CD66a-allophycocyanin (APC) (eBioscience), CD55-PE (BioLegend), CD11b-Alexa Fluor 700 (BioLegend). Samples were then run on a flow cytometer [53].

### 16. Multicolor Flow Cytometry.

SONY SA3800 flow cytometer was used to acquire the data. To validate the performance of the machine, Align check beads were used. An unstained sample was used to detect the autofluorescence signal and as a reference for the negative spectrum. For a reference for colors, single-color controls were used. To validate the gating strategy and antibody labeling FMO (fluorescence minus one) controls were used. At least 20,000 events were acquired per sample.

For human blood assay, Gating was performed by use of SSC-Area (SSC-A) by FSC-Area (FSC-A) gate for granulocytes followed by an SSC width (SSC-W) by SSC height (SSC-H) gate to remove doublets. To gate on Neutrophils three steps were followed: SSC-Area (SSC-A) by CD16hi, SSC-Area (SSC-A) by CD 18hi, and SSC-Area (SSC-A) by CD66hi. To gate on monocyte after removing the doublets, cells were gated using CD14hi by CD64hi.

For the bone marrow assay, SSC-Area (SSC-A) by FSC-Area (FSC-A) gate were used to gate on granulocytes followed by an SSC width (SSC-W) by SSC height (SSC-H) as well as FSC width (FSC-W) by FSC height (FSC-H) gate to remove doublets. Neutrophils were gated using SSC-Area (SSC-A) by F4/80 low followed by an SSC-Area (SSC-A) by Ly6G hi gate.

To gate on granulocyte in the Thp1 assay, SSC-Area (SSC-A) by FSC-Area (FSC-A) gate was used followed by an FSC width (FSC-W) by FSC height (FSC-H) gate for doublet removal. Live macrophages were then gated using SSC-Area (SSC-A) by eFlour 506low gate.

Data were analyzed by FlowJo software (v10.8; Tree Star). The mean fluorescence intensities (MFIs) for each CD marker were measured. Significant differences (P<0.05) were determined using GraphPad Prism 9 (GraphPad Software, Inc.). for the in-vitro human blood and mouse bone marrow assay paired Student's t-test (n=3) was used for the statistical analysis. For the Thp1 assay (n=3) one-way analysis of Variance (ANOVA) followed by a post hoc Tukey test was performed.

### 17. Cell Culture and Macrophages Work.

THP-1 cells; Acute Monocytic Leukemia; (Homo sapiens, ATCC TIB-202) were grown and subcultured in T-75 flasks using RPMI-1640 media supplemented with fetal bovine serum (final concentration 10%), 2-mercaptoethanol (final concentration 0.05 mM) and penicillin/streptomycin (final concentration 100 I.U./mL and 100 μg·ml⁻¹, respectively). Cells were kept at 37° C. with 5% CO₂ and maintained by replacing the media every 2 days. For the experiment, 8×10⁵ THP1 monocyte were seeded in a 6 well plate and differentiated into resting macrophages (M0) using 100 nM phorbol 12-myristate 13-acetate (PMA) (Sigma) for 24 hours at 37° C. with 5% CO₂. Macrophages were then polarized to M1 phenotype by adding 10 μg/ml of lipopolysaccharides (LPS) (Sigma) and 20 ng/ml of Interferon-gamma (IFN-γ) (PeproTech). Cells were then challenged with Salivaricin 10 (15 μg·ml⁻¹) for 24 hours at 37° C. with 5% CO₂, UltraPure Water (Invitrogen) was used as control. Cells were then rinsed with warm PBS and treated with 5 mM EDTA for 5 min at 37° C. with 5% CO₂. Cells were then collected and resuspended in 1 ml PBS, supplemented with 1 μl eFluor506, and incubated at 4° C. for 30 min. Cells were washed using fluorescent-activated cell sorting (FACS) buffer (Hanks' balanced salt solution, 1% bovine serum albumin, 2 mM EDTA) and labeled with the following antibodies: CD40 (AF647) BioLegend, CD163 (BV421) BioLegend, CD206 (PE) BioLegend. After 30 min of incubation on ice, cells were then washed with FACS buffer and fixed with 1.6% PFA for 15 minutes on ice. Cells were then resuspended in FACS buffer for flow cytometry analysis as mentioned above.

### 18. Blood Neutrophils Isolation

Five milliliter citrate sodium buffered human peripheral blood was layered on the top of 5 ml One step polymorphs in 15 ml centrifuge tube, spun 35 minutes with 500 RCF at room temperature. Neutrophils were recovered from the bottle layer. Mixed RBC was lysed with 1×BD pharm lyse. The total number of neutrophils are diluted with 1×PBS and counted with Beckman Coulter Z2 coulter.

### 19. Murine Neutrophil Preparations

Mice (8 to 16 week-old C57BL/6) were euthanized by CO₂ inhalation. Femurs and tibias were removed, and bone marrow was isolated. Bone marrow cells were layered onto discontinuous Percoll (Sigma, Oakville, ON, Canada) with gradients of 82, 65 and 55%. Mature neutrophils were recovered at the 82-65% interface.

### 20. Chemotaxis Assays

A suspension of 1×10⁶ human PMNs in 100 μL HBSS containing 1% gelatin (Sigma-Aldrich) was placed on a 5% bovine serum albumin-coated 22×40 mm glass coverslip and incubated for 10 minutes at 37° C. Coverslips were then inverted onto a Zigmond chamber. Hundred microliters of HBSS and 100 μL HBSS containing N-formyl-methionyl-leucyl-phenylalanine (fMLP) 10-6M or different concentrations of salivaricin 10 were added to the left and right chambers, respectively. PMN migration across the chamber was recorded with time-lapse of video microscopy at 20 second intervals for a period of 10 minutes using a Zeiss Motorized AxioVert (ZEISS). Captured images were analyzed using cell-tracking software (Retrac, v2.1.0.).

### 21. PMN Superoxide Production

A suspension of 2×10⁵ human blood neutrophil or murine bone marrow neutrophil in 20 μL of PBS was prepared in 96 well plate. A 176 μL final volume of PiCM-G buffer with 2 μL of equine ferricytochrome C was added to the suspension. The plate was incubated at for 10 minutes at 37° C. PMN stimulation was achieved through the addition of either 1 μM 2 μL of phorbol myristate acetate (PMA) or increasing concentration of salivaricin 10 peptide for 15 to 30 minutes at 37° C. The absorbance of reduced cytochrome c was monitored with a spectrophotometer at 550 nm.

### 22. Animal Trial

Mouse studies were performed in accordance with all relevant ethical regulations and were approved by the University of Toronto Animal Care Committee and the Research Ethics Board (Protocol #20010664). Peritonitis was induced by intraperitoneal injection of pHrodo-Red Escherichia coli BioParticles

(Molecular Probes) as described previously [54] followed by 100 μL injection of salivaricin 10 (50 μg) in PBS using the same method. All mice were 8 to 16 week-old C57BL/6 and were euthanized by CO₂ inhalation before injections and cell collection. After 3 hours, cells were retrieved from the peritoneal cavity by lavage with 3 mL cold PBS. Cell counts were obtained on a Coulter counter, and the counts were normalized to the volume recovered. Sample preparation for flow cytometry analysis was carried out as described above for in vitro experiments.

The purpose of the above description is to illustrate some configurations and uses of the present invention, without implying any limitation. It will be apparent to those skilled in the art that various modifications and variations may be made in the process and product of the invention without departing from the spirit or scope of the invention.

### References (all of which are Incorporated Herein by Reference Thereto)

1. Proctor, L., et al., A review of 10 years of human microbiome research activities at the US National Institutes of Health, Fiscal Years 2007-2016. Microbiome, 2019. 7(1): p. 31.
2. Bitschar, K., et al., Lugdunin amplifies innate immune responses in the skin in synergy with host-and microbiota-derived factors. Nature Communications, 2019. 10(1): p. 2730.
3. Caselli, E., et al., Defining the oral microbiome by whole-genome sequencing and resistome analysis: the complexity of the healthy picture. BMC Microbiology, 2020. 20(1): p. 120.
4. Barbour, A., et al., Metabolites of the oral microbiome: important mediators of multikingdom interactions. FEMS Microbiology Reviews, 2021.
5. Lamont, R. J. and G. Hajishengallis, Polymicrobial synergy and dysbiosis in inflammatory disease. Trends in Molecular Medicine, 2015. 21(3): p. 172-183.
6. Baker, J. L., et al., Ecology of the Oral Microbiome: Beyond Bacteria. Trends in Microbiology, 2017. 25(5): p. 362-374.
7. Radaic, A. and Y. L. Kapila, The oralome and its dysbiosis: New insights into oral microbiome-host interactions. Computational and Structural Biotechnology Journal, 2021. 19: p. 1335-1360.
8. Sulyanto, R. M., et al., The Predominant Oral Microbiota Is Acquired Early in an Organized Pattern. Scientific Reports, 2019. 9(1): p. 10550.
9. Kaci, G., et al., Anti-inflammatory properties of Streptococcus salivarius, a commensal bacterium of the oral cavity and digestive tract. Applied and environmental microbiology, 2014. 80(3): p. 928-934.
10. Barbour, A., P. Wescombe, and L. Smith, Evolution of Lantibiotic Salivaricins: New Weapons to Fight Infectious Diseases. Trends in Microbiology, 2020. 28(7): p. 578-593.
11. Arnison, P. G., et al., Ribosomally synthesized and post-translationally modified peptide natural products: overview and recommendations for a universal nomenclature. Natural Product Reports, 2013. 30(1): p. 108-160.
12. Mohr, K. I., et al., Pinensins: The First Antifungal Lantibiotics. 2015. 54(38): p. 11254-11258.
13. Fu, Y., A. H. Jaarsma, and O. P. Kuipers, Antiviral activities and applications of ribosomally synthesized and post-translationally modified peptides (RiPPs). Cellular and Molecular Life Sciences, 2021. 78(8): p. 3921-3940.
14. Prochnow, H., et al., Labyrinthopeptins Exert Broad-Spectrum Antiviral Activity through Lipid-Binding-Mediated Virolysis. 2020. 94(2): p. e01471-19.
15. Iorio, M., et al., A Glycosylated, Labionin-Containing Lanthipeptide with Marked Antinociceptive Activity. ACS Chemical Biology, 2014. 9(2): p. 398-404.
16. Meindl, K., et al., Labyrinthopeptins: A New Class of Carbacyclic Lantibiotics. 2010. 49(6): p. 1151-1154.
17. Staden, A. D. P. v., et al., Therapeutic Application of Lantibiotics and Other Lanthipeptides: Old and New Findings. 2021. 87(14): p. e00186-21.
18. Kindrachuk, J., et al., Manipulation of innate immunity by a bacterial secreted peptide: Lantibiotic nisin Z is selectively immunomodulatory. 2013. 19(3): p. 315-327.
19. Chatterjee, C., et al., Biosynthesis and Mode of Action of Lantibiotics. Chemical Reviews, 2005. 105(2): p. 633-684.
20. Repka, L. M., et al., Mechanistic Understanding of Lanthipeptide Biosynthetic Enzymes. Chemical Reviews, 2017. 117(8): p. 5457-5520.
21. Gerlt, J. A., et al., Enzyme Function Initiative-Enzyme Similarity Tool (EFI-EST): A web tool for generating protein sequence similarity networks. Biochimica et Biophysica Acta (BBA)-Proteins and Proteomics, 2015. 1854(8): p. 1019-1037.
22. Shannon, P., et al., Cytoscape: a software environment for integrated models of biomolecular interaction networks. Genome Res, 2003. 13(11): p. 2498-504.
23. Afonina, I., et al., The composition and function of Enterococcus faecalis membrane vesicles. microLife, 2021. 2.
24. Abranches, J., et al., Biology of Oral Streptococci. 2018. 6(5): p. 6.5.11.
25 Stingu, C.-S., et al., Periodontitis is associated with a loss of colonization by Streptococcus sanguinis. 2008. 57(4): p. 495-499.
26. Huffines, J. T. and J. A. Scoffield, Disruption of Streptococcus mutans and Candida albicans synergy by a commensal streptococcus. Scientific Reports, 2020. 10(1): p. 19661.
27. Shin, J. M., et al., Antimicrobial nisin acts against saliva derived multi-species biofilms without cytotoxicity to human oral cells. 2015. 6(617).
28. Fine, N., et al., Periodontal Inflammation Primes the Systemic Innate Immune Response. 2021. 100(3): p. 318-325.
29. Fine, N., et al., Distinct Oral Neutrophil Subsets Define Health and Periodontal Disease States. 2016. 95(8): p. 931-938.
30. Nguyen, G. T., E. R. Green, and J. Mecsas, Neutrophils to the ROScue: Mechanisms of NADPH Oxidase Activation and Bacterial Resistance. 2017. 7(373).
31. Dharmaprakash, A., et al., Development of broad-spectrum antibiofilm drugs: strategies and challenges. 2015. 10(6): p. 1035-1048.
32. Wang, M., et al., Microbial Hijacking of Complement-Toll-Like Receptor Crosstalk. 2010. 3(109): p. ra11-ra11.
33. Medina, E., et al., Survival of Streptococcus pyogenes within Host Phagocytic Cells: A Pathogenic Mechanism for Persistence and Systemic Invasion. The Journal of Infectious Diseases, 2003. 187(4): p. 597-603.
34. Buchanan, J. T., et al., DNase Expression Allows the Pathogen Group A Streptococcus to Escape Killing in Neutrophil Extracellular Traps. Current Biology, 2006. 16(4): p. 396-400.
35. Yamaguchi, M., et al., Streptococcus pneumoniae Evades Host Cell Phagocytosis and Limits Host Mortality Through Its Cell Wall Anchoring Protein PfbA. 2019. 9(301).
36. Barbour, A. and K. Philip, Variable Characteristics of Bacteriocin-Producing Streptococcus salivarius Strains Isolated from Malaysian Subjects. PLOS ONE, 2014. 9(6): p. e100541.
37. Hoshino, T., et al., PCR detection and identification of oral streptococci in saliva samples using GTF genes. Diagnostic Microbiology and Infectious Disease, 2004. 48(3): p. 195-199.
38. Wescombe, P. A., et al., Salivaricin G32, a Homolog of the Prototype Streptococcus pyogenes Nisin-Like Lantibiotic SA-FF22, Produced by the Commensal Species Streptococcus salivarius. International Journal of Microbiology, 2012. 2012: p. 738503.
39. Chin, C.-S., et al., Nonhybrid, finished microbial genome assemblies from long-read SMRT sequencing data. Nature Methods, 2013. 10(6): p. 563-569.
40. Overbeek, R., et al., The SEED and the Rapid Annotation of microbial genomes using Subsystems Technology (RAST). Nucleic Acids Research, 2013. 42(D1): p. D206-D214.
41. Blin, K., et al., antiSMASH 5.0: updates to the secondary metabolite genome mining pipeline. Nucleic Acids Research, 2019. 47(W1): p. W81-W87.
42. Geng, M., et al., Covalent Structure and Bioactivity of the Type All Lantibiotic Salivaricin A2. 2018. 84(5): p. e02528-17.
43. Ravichandran, A., et al., The Presence of Two Cyclase Thioesterases Expands the Conformational Freedom of the Cyclic Peptide Occidiofungin. Journal of Natural Products, 2013. 76(2): p. 150-156.
44. Wüthrich, K. J. E. N., NMR with Proteins and Nucleic Acids. 1986. 17(1): p. 11-13.
45. Braunschweiler, L. and R. R. Ernst, Coherence transfer by isotropic mixing: Application to proton correlation spectroscopy. Journal of Magnetic Resonance (1969), 1983. 53(3): p. 521-528.
46. Kumar, A., R. R. Ernst, and K. Wüthrich, A two-dimensional nuclear Overhauser enhancement (2D NOE) experiment for the elucidation of complete proton-proton cross-relaxation networks in biological macromolecules. Biochemical and Biophysical Research Communications, 1980. 95(1): p. 1-6.
47. Johnson, B. A. and R. A. Blevins, NMR View: A computer program for the visualization and analysis of NMR data. Journal of Biomolecular NMR, 1994. 4(5): p. 603-614.
48. Barbour, A., et al., New insights into the mode of action of the lantibiotic salivaricin B. Scientific Reports, 2016. 6(1): p. 31749.
49. Caporaso, J. G., et al., Ultra-high-throughput microbial community analysis on the Illumina HiSeq and MiSeq platforms. The ISME Journal, 2012. 6(8): p. 1621-1624.
50. Wang, Q., et al., Naive Bayesian Classifier for Rapid Assignment of rRNA Sequences into the New Bacterial Taxonomy. 2007. 73(16): p. 5261-5267.
51. Caporaso, J. G., et al., QUIME allows analysis of high-throughput community sequencing data. Nature Methods, 2010. 7(5): p. 335-336.
52. Price, M. N., P. S. Dehal, and A. P. Arkin, FastTree: Computing Large Minimum Evolution Trees with Profiles instead of a Distance Matrix. Molecular Biology and Evolution, 2009. 26(7): p. 1641-1650.
53. Oveisi, M., et al., Novel Assay To Characterize Neutrophil Responses to Oral Biofilms. 2019. 87(2): p. e00790-18.
54. Fine, N., et al., Primed PMNs in healthy mouse and human circulation are first responders during acute inflammation. Blood Advances, 2019. 3(10): p. 1622-1637.

1. A composition comprising a pre-SrnA1 peptide having a sequence of SEQ ID NO.: 1, a SrnA1 peptide having a sequence of SEQ ID NO: 2, a pre-SrnA2 peptide having a sequence of SEQ ID NO.3, a SrnA2 peptide having a sequence of SEQ ID NO: 4, a pre-SrnA4 peptide having a sequence of SEQ ID NO.: 7, a SrnA4 peptide having a sequence of SEQ ID NO: 8, a peptide having the sequence of SEQ ID NO: 16, or post-translationally modified versions thereof.

2-4. (canceled)

5. The composition of claim 1 wherein the post translational modifications comprises a phosphorylation at Thr4 amino acid of each or all of SrnA1, SrnA2 and SrnA4.

6. The composition of claim 4 wherein the post translational modifications comprise a single phosphorylation at Thr4 amino acid of each or all of SrnA1, SrnA2 and SrnA4.

7. (canceled)

8. The composition of claim 1 wherein the post-translational modifications comprise dehydrations at one or more of amino acids 9, 19 and 21 of each or all of SrnA1, SrnA2 and SrnA4.

9. The composition of claim 1 wherein the post-translational modifications comprise a β-methyllanthionine ring between amino acids 9 and 14 (Abu9-S-Ala14), a β-methyllanthionine ring between amino acids 19 and 24 (Abu19-S-Ala24), and a lanthionine ring between amino acids 21 and 31 (Ala21-S-Ala31) of each or all of SrnA1, SrnA2 and SrnA4.

10-11. (canceled)

12. The composition of claim 1 derived from a SALI-10 strain of S. salivarius.

13. The composition of claim 12 wherein the SALI-10 is characterized by comprising a megaplasmid.

14. The composition of claim 13 wherein the megaplasmid has a size of 164 kb.

15. The composition of claim 14 wherein the megaplasmid has a sequence of SEQ ID NO.: 15.

16. The composition of claim 12 wherein the SALI-10 is characterized by comprising a gene sequence of SEQ ID NO.: 14.

17. The composition of claim 11 wherein the SALI-10 comprises an SrnM lanthionine synthetase having a sequence of SEQ ID NO:9.

18. A pharmaceutical composition comprising the composition of claim 1 and a pharmaceutically acceptable excipient or carrier.

19. Method of treating a bacterial infection caused by a pathogen selected from the group consisting of multi-drug resistant (MDR) Streptococcus pneumoniae, Streptococcus pyogenes, Streptococcus dysagalactiae, vancomycin-resistant Enterococcus faecium, Porphyromonas gingivalis, Tannerella forsythia, and MDR Enterococcus faecalis comprising administering a pharmaceutically acceptable amount of the pharmaceutical composition of claim 18 to the patient.

20. (canceled)

21. Method of treating Actinomyces graevenitzii and/or Granulicatella adiacens-associated pulmonary abscess, Bifidobacterium dentium associated caries, or S. pyogenes associated strep throat in a patient in need thereof, or of modulating an anti-inflammatory response or reducing inflammation in a patient in need thereof, comprising administering a pharmaceutically acceptable amount of the pharmaceutical composition of claim 18 to the patient.

22-29. (canceled)

30. Method of modulating an anti-inflammatory response, modulating phagocytic activity of neutrophils, or activating reactive oxygen species of neutrophils in a patient in need thereof, comprising administering a pharmaceutically acceptable amount of salivaricin peptides to the patient.

31-32. (canceled)

33. Method of activating neutrophil chemotaxis, or polarizing macrophages towards M2 anti-inflammatory phenotype in a patient in need thereof, comprising administering a pharmaceutically acceptable amount of the pharmaceutical composition of claim 18 or a salivaricin peptide to the patient.

34-35. (canceled)

36. An isolated S. salivarius strain SALI-10 having: (a) a genome sequence of SEQ ID NO: 14; (b) a megaplasmid sequence of SEQ ID NO: 15; (c) an SrnM having a sequence of SEQ ID NO: 9; and (d) expressing a peptide SrnA1 having a sequence of SEQ ID NO: 2, a peptide SrnA2 having a sequence of SEQ ID NO:4, a peptide SrnA4 having a sequence of SEQ ID NO: 8, or post-translationally modified versions thereof.

37-40. (canceled)

41. The isolated S. salivarius of claim 36 wherein the post translational modifications comprises a phosphorylation at Thr4 of each or all of SrnA1, SrnA2 and SrnA4.

42. The isolated S. salivarius of claim 36 wherein the post translational modifications comprise a single phosphorylation at Thr4 of each or all of SrnA1, SrnA2 and SrnA4.

43. The isolated S. salivarius of claim 36 wherein the post-translational modifications comprise three dehydrations and one phosphorylation.

44. The isolated S. salivarius of claim 43 wherein the post-translational modifications consist of three dehydrations and one phosphorylation.

45-46. (canceled)
